SEQUENCE LISTING (13 sequences)

SEQ ID NO: 1 - protein, 581 aa, Adenovirus serotype 5
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 2 - DNA, 42 bp, Artificial Sequence
  Description of Artificial Sequence Synthetic oligonucleotide
gcatttagtctacagttaggctctggagctggtgtggtccac

SEQ ID NO: 3 - DNA, 39 bp, Artificial Sequence
  Description of Artificial Sequence Synthetic oligonucleotide
gttaggcataaatccaacgtcgtttgtataggctgtgcc

SEQ ID NO: 4 - DNA, 39 bp, Artificial Sequence
  Description of Artificial Sequence Synthetic oligonucleotide
accgtgagattttggatagtctgataggttaggcataaa

SEQ ID NO: 5 - DNA, 47 bp, Artificial Sequence
  Description of Artificial Sequence Synthetic oligonucleotide
ctacagttaggagatggagcgggcccggtccacaaagttagcttatc

SEQ ID NO: 6 - DNA, 39 bp, Artificial Sequence
  Description of Artificial Sequence Synthetic oligonucleotide
gtctacagttaggagatggctttggtgtggtccacaaag

SEQ ID NO: 7 - DNA, 33 bp, Artificial Sequence
  Description of Artificial Sequence Synthetic oligonucleotide
aagatgagcactttgctttgttccagatattgg

SEQ ID NO: 8 - DNA, 39 bp, Artificial Sequence
  Description of Artificial Sequence Synthetic oligonucleotide
gtggccagaccagtcccacttaaatgacatagagtatgc

SEQ ID NO: 9 - DNA, 45 bp, Artificial Sequence
  Description of Artificial Sequence Synthetic oligonucleotide
acttttggcagttttacccttagactgtggataagctgataggtt

SEQ ID NO: 10 - DNA, 60 bp, Artificial Sequence
  Description of Artificial Sequence Synthetic oligonucleotide
aacgattctttagctgccgggagcagaggcggaggcggaggcgctgggttcttgggcaat

SEQ ID NO: 11 - DNA, 58 bp, Artificial Sequence
  Description of Artificial Sequence Synthetic oligonucleotide
cacaaacgatctttacttcttcttttttcttctttttggatccgggagcgaggcggag

SEQ ID NO: 12 - DNA, 26 bp, Artificial Sequence
  Description of Artificial Sequence Synthetic oligonucleotide
tgaaaaatgattcgaaattttctgca

SEQ ID NO: 13 - protein, 12 aa, Artificial Sequence
  Description of Artificial Sequence Synthetic linker peptide
PSASASASAPGS

(19) United States
(12) Patent Application Publication
(10) Pub. No.: US 2006/0228334 A1
(43) Pub. Date: Oct. 12, 2006

(54) Modified adenoviral fiber with ablated to cellular receptors

(76) Inventors: Manuel Rosa-Calatrava, Strasbourg (FR); Philippe Leissner, Caluire Et Cuire (FR); Valerie Legrand, Illkirch Graffenstaden (FR)

Correspondence Address: Burns Doane;Swecker & Mathis, PO Box 1404, Alexandria, VA, 22313-1404, US

(21) Appl. No.: 10/520,626

(22) PCT Filed: Jul. 10, 2003
(86) PCT No.: PCT/IB03/03336

§ 371 Date: Jan. 10, 2005

Related U.S. Application Data
(60) Provisional application No. 60/409,228, filed on Sep. 10, 2002.

(30) Foreign Application Priority Data
Jul. 10, 2002 (EP) 02360204.8

(51) Int. Cl. A61K 48/00; C12N 15/861; C12N 7/00

(52) U.S. Cl. 424/93.2; 435/235.1; 435/456

(57) ABSTRACT

The present invention concerns a modified adenoviral fiber containing at least one mutation affecting one or more amino acid residue(s) of said adenoviral fiber interacting with at least one glycosaminoglycan and/or sialic acid-containing cellular receptor, as well as a trimer of such a modified adenoviral fiber. The present invention also relates to a DNA fragment, an expression vector encoding said modified adenoviral fiber. The present invention also concerns an adenoviral particle lacking a wild-type fiber and comprising the trimer of modified adenoviral fibers as well as a process for producing such an adenoviral particle. Theto present invention also provides a composition comprising such an adenoviral particle and the therapeutic use thereof.

The present invention relates to an adenoviral fiber protein mutated in the region(s) or residue(s) involved in recognizing and/or binding to at least one cell-surface glycosaminoglycan or sialic acid-containing receptor. It also relates to an adenovirus particle bearing at its surface such a mutated fiber, having a reduced or ablated capacity to interact with such glycosaminoglycan or sialic acid-containing receptors. The present invention also provides an adenoviral fiber protein mutated in the region(s) or residue(s) involved in recognizing and binding to both such glycosaminoglycan or sialic acid-containing receptors and to the coxsackie-adenovirus receptor (CAR). It also relates to an adenovirus particle bearing at its surface such a doubly mutated fiber, having a reduced or ablated capacity to interact with both CAR and such glycosaminoglycan and/or sialic acid-containing cellular receptors. Such adenovirus particles can optionally be combined with a ligand which confers modified or retargeted host specificity. The invention is of most particular value in the context of adenovirus targeting and the development of targeted vectors that can be used for multiple gene therapy applications, including cancer, cardiovascular, genetic, and inflammatory diseases.

Adenoviruses have, been detected in many animal species, are non-integrative and low pathogene. They are able to infect a variety of cell types, dividing as well as quiescent cells. They have a natural tropism for airway epithelia. In addition, they have been used as live enteric vaccines for many years with an excellent safety profile. Finally, they can be easily grown and purified in large quantities. These features have made recombinant adenoviruses particularly appropriate for use as gene therapy vectors for a large variety of therapeutic and vaccine applications.

Adenoviral genome consists of a linear double-standed DNA molecule of approximately 36 kb (conventionally divided into 100 map units (mu)) carrying more than about thirty genes necessary to complete the viral cycle. During productive adenoviral infection, three classes of viral genes are temporally expressed in the following order: early (E), intermediate and late (L). The early genes are divided into 4 regions dispersed in the adenoviral genome (E1 to E4). The E1, E2 and E4 regions being essential to viral replication whereas E3 region is dispensable in this respect. The E1 region (E1A and E1B) encodes proteins responsible for the regulation of transcription of the viral genome. Expression of the E2 region genes (E2A and E2B) leads to the synthesis of the polypeptides needed for viral replication (Pettersson and Roberts, 1986, In Cancer Cells (Vol 4): DNA Tumor Viruses, Botchan and Glodzicker Sharp Eds pp 37-47, Cold Spring Harbor Laboratory, Cold Spring Harbor, N.Y.). The proteins encoded by the E3 region prevent cytolysis by cytotoxic T cells and tumor necrosis factor (Wold and Gooding, 1991, Virology 184, 1-8). The proteins encoded by the E4 region are involved in DNA replication, late gene expression, splicing and host cell shut off (Halbert et al., 1985, J. virol. 56, 250-257). The late genes (L1 to L5) are mostly transcribed from the major late promoter (MLP). They overlap at least in part with the early transcription units and encode in their majority the structural proteins constituting the viral capsid. In addition, the adenoviral genome carries at both extremities cis-acting regions essential for DNA replication, respectively the 5′ and 3′ ITRs (Inverted Terminal Repeats) which harbor origins of DNA replication and the packaging sequence immediately adjacent to the 5′ITR.

Most of the adenoviral vectors presently used in gene therapy protocols are replication-defective viruses (i.e. incapable of dividing or proliferating in the host cells they infect), to avoid their dissemination in the environment and the host organism. The feasability of gene transfer using E1-deleted vectors has been demonstrated into a variety of tissues in vivo (see for example Yei et al., 1994, Hum. Gene Ther. 5, 731-744; Dai et al., 1995, Proc. Natl. Acad. Sci. USA 92, 1401-1405; Howell et al., 1998, Hum. Gene Ther. 9, 629-634; Nielsen et al., 1998, Hum. Gene Ther. 9, 681-694). However, their use is associated with acute inflammation and toxicity in a number of animal models (Yang et al., 1994, Proc. Natl. Acad. Sci. USA 91, 4407-4411; Zsengeller et al., 1995, Hum. Gene Ther. 6, 457-467) as well as with host immune responses to the viral vector and gene products (Yang et al., 1995, J. Virol. 69, 2004-2015), resulting in the elimination of the infected cells and transient gene expression. Second-generation adenovirus vectors having additional viral genes deleted to overcome adenovirus-mediated immunogenicity are currently investigated (Engelhardt et al., 1994, Hum. Gene Ther. 5, 1217-1229; Engelhardt et al., 1994, Proc. Natl. Acad. Sci. USA 91, 6196-6200). Evaluation of E1 and partially E4-deleted adenoviral vectors in vivo have shown a reduced hepatotoxicity and inflammation (Christ et al., 2000, Human Gene Ther. 11, 415-427).

The initial attachment of the adenovirus particle to the cell surface is mediated by the binding of the knob region of the viral fiber protein to ubiquitous cell surface receptors. Two distinct proteins belonging to the immunoglobulin superfamily were reported as the primary receptors for adenovirus serotype C fibers: the coxsackievirus-adenovirus receptor (termed CAR) (Bergelson et al, 1997, Science 275, 1320-1323. Tomko et al., 1997, Proc. Natl. Acad. Sci. USA 94, 3352-3356) and the alpha 2 domain of the major histocompatibility complex class I molecule (Hong el al., 1997, EMBO J. 16, 2294-2306). A predominant role for CAR in adenovirus tropism is however suggested by the work of McDonald et al. (1999, Gene Ther. 6, 1512-1519), who demonstrated discordance between MHC class I heavy chain levels at the cell surface and adenovirus susceptibility. In addition to subgroup C adenoviral fibers, CAR was also shown to bind to subgroups A, D, E and F fibers (Roelvink et al., 1998, J. Virol. 72, 7909-7915) but not to subgroup B adenoviral fibers, such as those of serotype 3 and 7 (Krasnykh et al., 1996, J. Virol. 70, 6839-6846; Santis et al., J. Gen Virol. 80, 1519-1527).

More recently, cell-surface heparan sulfate glycosaminoglycans (HSG) were shown to interact with adenovin's serotype 5 (Ad5), which suggests that these molecules may also facilitate virus binding to cells (Decheccili et al., 2000, Virology 268, 382-390; Dechecchi et al., 2001, J. Virol. 75, 8772-8780).

Internalization into the cell of the attached adenoviral particles is mediated by the recognition of the Arg-Gly-Asp (RGD) sequence located in the viral penton base protein by the cellular alphav integrins (Mathias et al., 1994, J. Virol. 68, 6811-6814). This interaction triggers cellular internalization whereby: the virions achieve localization within the endosome. Acidification of the endosome elicits conformation changes in the capsid proteins, allowing their interaction with the endosome membrane in a manner that achieves vesicle disruption and particle escape. Following endosomolysis, the virion translocates to the nucleus, where the subsequent steps of the viral life cycle occur.

The almost ubiquitous distribution of the CAR cellular receptor is thought to be primarily responsible for the broad cell tropism of the human serotype C adenoviruses. Consistent with this notion, the absence or reduced expression of this receptor has been shown to correlate with the poor sensitivity of certain cell types (e.g. lymphocytes, smooth muscle cells) to adenovirus transduction (Leon et al, 1998, Proc. Natl. Acad. Sci. USA 95, 13159-13164; March et al., 1995, Hum. Gene Ther. 6, 41-63). Moreover, numerous studies have now reported that primary tumor cells express only low levels of CAR (Li et al., 1999, Cancer Res. 59, 325-330; Miller et al., 1998, Cancer Res, 58, 5738-5748).

The ability of adenoviruses to mediate infection of a broad spectrum of dividing and non-dividing cell types constitutes an advantage over alternative gene transfer vectors. However, this broad tissue tropism may also turn disadvantageous when genes encoding potentially harmful proteins (e.g. cytokines, cytotoxic proteins, suicide gene products) are expressed in surrounding normal tissues. Moreover, the overall in vitro efficiency of gene delivery might be reduced by a significant dilution of the virus in the organism due to the transduction of non-target cells. The development of adenovirus vectors with defined targeted entry pathways would therefore greatly improve the safety and efficacy of some current gene therapy strategies. Thus, targeting adenoviral vectors may improve gene therapy procedures by either enhancing infectivity to transduction refractory cells (e.g. primary tumor cells) or restricting the viral tropism to specific tissue(s) of interest.

In this regard, increasing efforts have been made during the last years to redirect the adenovirus tropism from its natural receptors to specific cell surface molecules. Since interactions of fiber and penton base with their corresponding cellular receptors represent key determinants of the viral tropism, retargeting the adenovirus may in principle be achieved by genetically, immunologically or chemically altering the capsid proteins (see for example WO94/10323 and for a review Barnett et al., 2002, Biochemica et Biophysica Acta 1575, 1-14). Such modifications aim to abolish the interaction of the virus with its natural receptors and to provide new ligands recognizing molecules specifically expressed on the targeted cells.

The Ad5 fiber protein is a long trimeric protein that protrudes from the virion surface. Each fiber monomer consists of three regions: the tail which associates with the penton base protein, the shaft, the length of which varies among various serotypes and is characterized by a repeating motif of approximately 15 residues (Green et al., 1983, EMBO J. 2, 1357-1365 Signas et al., 1985; J. Virol. 53, 672-678), and the knob which interacts with the cellular receptors (Henry et al., 1994, J. Virol. 68, 5239-5246). In Ad2, the C-terminal 40 aa residues in the knob and the last shaft repeat are required for Ad2 fiber trimerization (Hong and Engler, 1996. J. Virol. 70, 7071-7078; Novelli and Boulanger, 1991, Virol. 185, 365-376).

The crystal structure of the Ad5 fiber knob has been determined from protein expressed in bacteria. It is a trimer with a three-bladed propeller and a surface depression. Each knob monomer is organized as an eight-stranded antiparallel beta-sheet structure with loops and tunas connecting the beta-sheets (Xia et al., 1994, Structure 2, 1259-1270). Four of the beta-sheets (C, B, A and J) constitute the V-sheet: which faces towards the virion. The four other beta-sheets (G, H, I and D) form the R sheet and are presumed to face the cellular receptor. The V sheet seems to play an important role in the trimerization of the fiber structure, while the R sheet is thought to be involved in the interaction with the receptor.

Recently, specific mutations which eliminate the interaction with CAR were identified, demonstrating that the CAR binding site of the fiber knob domain can be mutated without adversely affecting the quaternary structure and overall conformation of the purified recombinant protein (WO98/44121, WO01/16344 and WO01/38361). For example, fiber proteins carrying amino acid substitutions in the AB loop (involving Ser408 and Pro409), in the DG loop (e.g. involving Tyr 477, Tyr 491, Ala 494 or Ala 503) and in beta-strand F (e.g. involving Leu 485) or having deletion of two consecutive amino acid in the DG loop were shown to alter CAR binding (Bewley et al., 1999, Science 286, 1579-1583; Kirby et al., 1999, J. Virol 73, 9508-9514; Kirby et al., 2000, J. Virol. 74, 2804-2813; Leissner et al., 2001, Gene Ther. 8, 49-57). Extending these data, it has been shown that viable and fully maturated viruses, carrying trimeric fibers mutated in the CAR binding domain, can be generated (Leissner et al., 2001, Gene Ther. 8, 49-57; Roelvink et al., 1999, Science 286, 1568-1571; Jakubczak et al., 2001, J. Virol. 75, 2972-2981). These viruses are structurally identical to native viruses and therefore constitute appropriate substrates for the insertion of targeting ligands in the mutated fibers. In this respect, some specific locations in the fiber protein have been identified for incorporation of a novel targeting ligand into the fiber knob domain.

For example, addition of 24 amino acids containing the Gastrin Releasing Peptide at the C-terminal end of the fiber did not prevent fiber trimerization (Michael et, al., 1995, Gene Ther. 2, 660-668). Similarly, addition at the same location of peptides of various lengths (17, 21 or 32 amino acids) was shown to yield viable viruses (Wickham et al., 1997, J. Virol 71, 8221-8229). Several groups have reported that insertion of stretches of lysine residues at the C-terminal end of the knob could lead to the generation of high titer viruses that were characterized by a 10 to 300 fold increase in their efficiency of infection of CAR-deficient cells, such as macrophages, endothelial cells, smooth muscle cells or T lymphocytes (Wickham et al., 1997, J. Virol 71, 8221-8229; Yoshida et al., 1998, Hum. Gene Ther. 9, 2503-2515; Wickham et al., 1996, Nature Biotechnology 14, 1570-1573; Bouri et al., 1999, Hum. Gene Ther. 10, 1633-1640).

Apart from the carboxy-terminal end of the fiber, Krasnykh et al. demonstrated that the HI loop in the knob domain could be used to successfully insert targeting ligands up to at least 63 amino acids without altering viral viability (Krasnykh et al., 1998, J. Virol. 72, 1844-1852; Krasnykh et al., 2000, Cancer Res. 60, 6784-6787). For instance, insertion of a RGD motif in the HI loop was shown to expand the tropism of the vector via the utilization of a CAR-independent cell entry mechanism (Dmitriev et al., 1998, J. Virol. 72, 9706-9713), allowing an enhancement in gene delivery to different primary tumors. Furthermore, the addition of this motif into the HI loop was shown to alter the transgene expression profile of systemically administered vector, with a reduction of liver expression and simultaneous increase in the lung, heart and spleen (Reynolds et al., 1999, Gene Ther. 6, 1336-1339). The introduction of a peptide ligand binding the transferrin receptor in the HI loop facilitated gene transfer to cells which over-express this receptor (Xia et al., 2000, J. Virol. 74, 11359-11366). Similarly, a HUVEC cell-binding peptide allowed a significant increase of the transduction efficiency of the retargeted vector towards these cells which are normally refractory to transduction (Nicklin et al., 2000, Circulation 102, 231-237).

Such mutated adenoviral vectors show reduced transduction of CAR-expressing cells in vitro but retain significant CAR-independent infectivity in vivo. It has been presumed that residual transduction could be mediated through interaction between the adenoviral penton base protein and cellular integrins. More recently, doubly ablated adenoviral vectors, lacking both CAR and integrin binding capacities were proposed to abolish adenovirus native tropism (Einfeld et al., 2001, J. Virol. 75, 11284-11291; Van Beusechem et al., 2001, J. Virol. 76, 2753-2762).

Thus, the prior art is deficient in mutated adenoviral fiber proteins that allow for reduction of the interaction with alternative cellular receptors (other than CAR and integrins) which are involved in adenovirus attachment or internalization, and especially with the newly identified primary receptor for adenovirus, heparan sulfate glycosaminoglycans (HSG). The present invention fullfills this long-standing need and desire in the art.

Therefore, the present invention provides novel mutants of the adenoviral fiber which allow, in particular, the production of viral particles having the following properties:
(i) the adenoviral particles comprising said modified fiber lack or substantially exhibit a substantially reduced binding to at least sialic acid-containing receptors and/or glycosaminoglycan-containing receptors, such as heparin/heparan sulfate-containing receptors, and more particularly to HSG receptors. The host specificity of these adenoviral particles bearing the modified fiber is decreased or even inhibited, in comparison to the host specificity of the adenoviral particles carrying a nonmutated (i.e. wild-type) fiber.
(ii) When the mutated adenoviral particles also comprises mutation(s) abolishing CAR binding, the adenoviral particles comprising said doubly mutated fiber lack or exhibit a substantially reduced binding to both the CAR receptor and the sialic acid and/or glycosaminoglycan-containing receptors, such as heparin/heparan sulfate-containing receptors, and more particularly to HSG receptors. Altering interactions with both CAR and HSG receptors may be essential to significantly restrict the native tropism of an adenoviral. Such a particle represents the best candidate for a basic vector that could be redirected by incorporation of specific targeting ligands.
(iii) When the adenoviral particle comprising said modified fiber also comprises a ligand specific for a cell-surface anti-ligand (e.g. a tumor-specific or tissue-specific antigen), it is possible to confer a novel tropism for one or more specific cell types exhibiting at its (their) surface said anti-ligand, in comparison to the nonmutated adenoviral particles.

The present invention has, in particular, the advantage of providing novel adenoviral particles, the properties of which make it possible to decrease the therapeutic amount of adenoviral particles to be administered, to reduce dilution in the host organism and to target the viral infection to the cells to be treated. This host specificity is particularly essential when an adenoviral vector expressing a cytotoxic gene is used, in order to avoid the propagation of the cytotoxic effect to healthy and nontargeted cells/tissues. In addition, the teachings of the present invention, allow other targeting systems intended for developing methods of treatment relying on recombinant viral and nonviral vectors.

Other and further aspects, features and advantages of the present invention will be apparent from the following description of the presently preferred embodiments of the invention. These embodiments are given for the purpose of disclosure.

Accordingly, the present invention relates to a modified adenoviral fiber containing at least one mutation affecting one or more amino acid residue(s) of said adenoviral fiber interacting with at least one glycosaminoglycan and/or sialic acid-containing cellular receptor.

The term “and/or” whereever used herein includes the meaning of “and”, “or” and “all or any other combination of the elements connected by said term”.

The term “about” or “approximately” as used herein means within 20%, preferably within 10%, and more preferably within 5% of a given value or range.

The term [symbol] amino acid [symbol] and residues are synonyms. This term refers to natural, unnatural and/or synthetic amino acids, including D or L optical isomers, modified amino acids and amino acid analogs.

The term [symbol] mutation [symbol] refers to a deletion, substitution or addition of one or more residues, or any combination of these possibilities. When several mutations are contemplated, they can concern consecutive residues and/or non consecutive residues. Mutation can be made in a number of ways known to those skilled in the art using recombinant techniques, including enzymatically cutting from the fiber-encoding sequence followed by modification and ligation of defined fragment, or by site-directed mutagenesis, especially by the Sculptor™ in vitro mutagenesis system (Amersham, Les Ullis, France) or by PCR techniques. Deletion mutation can comprise from about 1 to 20 amino acid residues, preferably not exceeding 11 amino acids. Deletion of one to three amino acids are preferred. According to a preferred embodiment, the mutation is a substitution of at least one amino acid residue by another. It is preferred that the mutation alters the charge of the substituted amino acid residue.

The [symbol] adenoviral fiber [symbol] as used herein refers to the structural protein present at the surface of an adenoviral capsid (also called pIV), which is known to mediate the early contact between virus and cells. The present invention encompasses the full length adenoviral fiber which is encoded by the complete coding sequence (i.e. from the initiator ATG codon to the stop codon). However, it is possible to employ a fragment thereof generated by internal deletion, or truncation having the properties as described herein. For illustrative purpose, the fiber-encoding sequence can be isolated from an adenoviral genome by conventional recombinant techniques. The fiber gene is present at the right end of the adenoviral genome positioned between E3 and E4 regions, e.g. from nucleotide (nt) 31042 to nt 32787 in the Ad5 genome and from nt 31030 to nt 32778 in the Ad2 genome.

The modified adenoviral fiber of the invention may originate (be obtained) from an adenovirus of human or animal origin (e.g. canine, avian, bovine, murine, ovine, porcine, feline, simien and the like) or be an hybrid comprising fragments of diverse origins. For instance, the adenovirus can be of subgroup A (e.g. serotypes 12, 18, 31), subgroup B (e.g. serotypes 3, 7, 11, 14, 16, 21, 34, 35, 50), subgroup C (e.g. serotypes 1, 2, 5, 6), subgroup D e.g. serotypes 8, 9, 10, 13, 15, 17, 19, 20, 22-30, 32, 33, 36-39, 42-47, 51), subgroup E serotype 4), subgroup F (serotype 40, 41), or any other adenoviral serotype. Preferably, however, the modified fiber of the invention originates from an adenovirus of subgroup C, with a special preference for Ad2 or Ad5 serotype.

The fiber of various human and animal adenoviruses are available on databases (e.g. GenBank) and literature publications. By way of information, mention is made of the GenBank and literature references for the fiber sequence of human serotype 2 (AAA92223), 3 (CAA26029). 5 (M 18369), 31 (CAA54050), 41 (X17016), 50 and 51 (De Jong et al., 1999, J. Clinical Microbiology 37, 3940), bovine BAV-3 (AF030154; see also WO98/59063 and Reddy et al., 1998, J. Virol. 72, 1394-1402) and canine CAV-2 (Rasmussen et al., 1995, Gene 159,279-280).

The fiber of Ad2 includes 582 amino acids (aa), the sequence of which being disclosed in Herissé et al. (1981, Nucleic Acid Res. 9, 4023-4042; incorporated into the present application by reference). The Ad5 fiber sequence was determined by Cliroboczek and Jacrot (1987, Virology 161, 549-554; incorporated by reference), and is 581 amino acids long including the initiator Met residue (as shown in SEQ ID NO: 1).

The crystal structure of the knob domain of the Ad5 fiber was determined by Xia et al. (1994, Structure 2, 1259-1270; incorporated by reference). For the purposes of the invention, the terms <<beta sheet and [symbol] loop [symbol] is as defined in Xia et al (1994). These terms are conventional in the field of protein biochemistry, and are defined in fundamental works (see for example Stryer, Biochemistry, 2^{nd} edition, Chap 2, p 11-39, Ed Freeman and Company, san Francisco). More specifically, each knob monomer includes 8 antiparallel beta sheets referred to as A to D and G to J, and 6 major loops of 8 to 55 residues. For example, loop AB connects beta sheet A to beta sheet B. It is indicated that minor sheets E and F are Considered to form part of loop DG connecting beta sheets D and G. By way of indication, Table 1 gives the location of these structures in the amino acid sequence of the wild-type Ad5 fiber, as shown in SEQ ID NO: 1, the +1 representing the Met initiator residue.

TABLE 1

| | |
|---|---|
| beta sheet | loop |

| nomenclature | Residues | Nomenclature | residues |
|---|---|---|---|
| A | 400 to 403 | AB | 404 to 418 |
| B | 419 to 428 | — | — |
| C | 431 to 440 | CD | 441 to 453 |
| D | 454 to 461 | DG | 462 to 514 |
| G | 515 to 521 | GH | 522 to 528 |
| H | 529 to 536 | HI | 537 to 549 |
| I | 550 to 557 | IJ | 558 to 572 |
| J | 573 to 578 | | |

In order to simplify the presentation of the present application, only the positions relating to Ad5 are specifically given. However, it is within the scope of those skilled in the art to adapt the present invention to other adenovirus fibers.

The one or more mutation(s) contained in the modified adenoviral fiber of the invention can affect amino acid residue(s) located in the tail, shaft and/or knob domains, with a special preference for the knob. The modified fiber of the invention preferably comprises a tail, a shaft and a knob. The various fiber region can be of the same serotype. Alternatively, it is also possible to use a [symbol] chimeric [symbol] fiber protein. For example, the tail and the shaft can be of one serotype (e.g. of a subgroup C adenovirus such as Ad2 or Ad5) and the knob can be of another serotype (e.g. of a subgroup B adenovirus such as Ad3 or Ad7).

Within the context of the present invention, the term [symbol] glycosaminoglycan-containing cellular receptor [symbol] encompasses any cell-surface molecule consisting of a core protein containing one or more covalently linked glycosaminoglycan side chains (e.g. linear side chains to form a long filament of glycosaminoglycan). The term [symbol] glycosaminoglycan [symbol] is conventional in the field of the art and can be defined as comprising disaccharide repeating units containing a derivative of an amino sugar, either glucosamine or galactosamine, with at least one of the sugars in the repeating units having a negatively charged carboxylate or sulfate group. Suitable glycosaminoglycan include without limitation chondritin sulfate, keratan sulfate, heparin, heparan sulfate, dermatan sulfate and hyaluronate as well as their various isoforms.

Preferably, the glycosaminoglycan-containing cellular receptor is a heparin- or heparan sulfate-containing cellular receptor. The structure of Heparin and heparan sulfate is for example illustrated in FIG. 18-15 of Biochemistry (4^{th} edition, Lubert Stryer; ed Freeman and Compagny, New York) and can be defined as a copolymer of glucosamine and glucuronic or iduronic acid with various sulfatations and/or acetylation modifications. Heparan sulfate is like heparin except that it has fewer N- and O-sulfate groups and more N-acetyl. Heparin- and heparan sulfate-containing cellular receptors have been widely illustrated in the literature, for example in Liu and Thorp (2002, Medical Research Reviews 22(1), 1-25; incorporated into the present application by reference). They are involved in many biological processes (e.g. blood coagulation, would healing, embryonic development, viral infections, etc.). The structure and the saccharide side chains of the various heparan sulfates-containing receptors encompassed by the present invention can vary according to their tissue distribution or their biological activities. Such heparan sulfate-containing receptors can be identified by conventional techniques in the art, combining techniques from virology, carbohydrate biochemistry, molecular biology and mass spectrometry.

In one preferred embodiment, the heparin or heparan sulfate-containing receptor encompassed by the present invention is the heparan sulfate glycosaminoglycan (HSG) receptors which normally interact with the wild-type adenoviral fiber, to mediate adenovirus attachment to a host cell. The HSG receptor is as defined in Dechecchi et al. (2000, Virology 268,382-390 and 2001, J. Virol. 75, 8772-8780).

Within the context of the present invention, the term [symbol] sialic acid-containing cellular receptor [symbol] encompasses any cell-surface molecule consisting of a core protein containing one or more polysaccharide side chains, such polysaccharide including sialic acid. Of course such receptors can exhibit complex pattern of glycosylation, containing apart sialic acid additional and diverse carbohydrate residues. The term [symbol] sialic acid [symbol] (also designated N-acetyl neuraminate) is conventional in the field of the art and denotes a 9 carbone sugar with a carboxylate group, which formula is for example given in FIG. 18-18 of Biochemistry (4^{th} edition, Lubert Stryer; ed Freeman and Compagny, New York)

Any native amino acid residue mediating or assisting in the interaction between the fiber and a native glycosaminoglycan-containing cellular receptor (more particularly the HSG receptor) and/or a sialic acid-containing cellular receptor is suitable for mutation. For example, the native amino acid residue(s) to modify may be involved in a conformational change associated with receptor binding. Alternatively, the mutation may result in a charge modification, a modification of a particular chemical group or a post-translational modification which alter the binding to the cellular receptor. The modified fiber of the present invention can be mutated at any number of such native amino acid residues, so long as it retains its ability to trimerize. The amino acid residue to be mutated can be within any region of the fiber (e.g. shaft and/or knob), and as far as the knob is concerned, within a beta sheet or within a loop connecting two beta sheets. It is also possible to replace one or more residue(s) of a fiber originating from a first adenovirus, said residue(s) mediating directly or indirectly binding to a native glycosaminoglycan (e.g. HSG), or sialic acid-containing receptor with equivalent residue(s) originating from a fiber of a second adenovirus not capable of interacting with such glycosaminoglycan or sialic acid-containing receptors.

Native amino acid residue to be mutated can be selected by any method in the art. For example, the sequences from different adenoviral serotypes (which are known in the art) can be compared to deduce conserved residues likely to mediate binding to glycosaminoglycan or sialic acid-containing cellular receptors, and more particularly to HSG receptors. Alternatively, or in combination, the sequence can be mapped on three dimensional representation of the protein to deduce those residues which are most likely responsible for such a binding. These analysis can be aided by resorting to any common algorithm or program for deducing protein structural function interaction. Alternatively, random mutation can be introduced into a cloned adenoviral fiber expression cassette (e.g. by site-directed mutagenesis, PCR amplification by varying the concentration of divalent cations in the PCR reaction, the error rate of the transcripts can be largely predetermined as described in Weiss et al., 1997, J. Virol. 71, 4385-4394 or Zhou et al., 1991, Nucleic Acid Res. 19, 6052). The mutated sequence then can be subcloned back in the template vector, thus generating a library of fibers, some of which will harbor mutations which diminish binding to glycosaminoglycan (e.g. HSG) and/or sialic acid-containing cellular receptors.

According to a preferred embodiment, the modified adenoviral fiber of the present invention has an affinity for said glycosaminoglycan and/or sialic acid-containing cellular receptor of at least about one order of magnitude less than a wild-type adenoviral fiber. The decrease or abolition of binding to glycosaminoglycan or sialic acid-containing cellular receptors, and in particular to the HSG receptors, can be evaluated by measuring infectivity or cell attachment provided by the modified fiber of the invention or virus particles harboring such a modified fiber, using the technique in the art. Monitoring can be autoradiography (e.g. employing radioactive viruses or radiolabeled fiber proteins), immunochemistry, or by measuring plaque formation, cytotoxicity or by evaluating gene delivery (e.g. using a reporter gene). For instance, suitable techniques include infection experiments of suitable cells carried out in the presence and in the absence of a competitor (i.e. heparin in the context of heparin/heparan sulfate-containing receptors or sialic acid in the context of sialic acid-containing receptors as described in the Experimental Section of the present application). For example, an adenovirus deficient or altered for HSG binding will be less or not competited by the competitor as compared to a wild-type adenovirus for infection of HSG-expressing cells. Indeed, after incubation of wild type adenovirus particles with heparin, the HSG-mediated pathway is inhibited due to the saturation of the wild type fiber with the competitor, whereas the infectivity of particles displaying a modified fiber of the invention is not substantially modified by the competitor. The alteration of the natural specificity can also be studied by evaluation of cell attachment using radiolabeled viruses (for example labeled with ³H thymidine, as described in Roelvin et al., 1996, J. Virol. 70, 76.14-7621) or radiolabeled fibers recombinantly produced. Alternatively, the affinity of the modified fiber of the invention can also be assayed for its ability to bind a substrate (e.g. heparin in the context of heparin/heparan sulfate-containing receptors or sialic acid in the context of sialic acid-containing receptors) immobilized oil an appropriate support using the Biacore technique. It is also possible to evaluate infectivity after pretreatment of suitable cells by heparinase (in the context of heparin/heparan sulfate-containing receptors) or sialidase (in the context of sialic acid-containing receptors).

The ability of the modified fiber of the present invention to bind to glycosaminoglycan (e.g. HSG receptors) or sialic acid-containing cellular receptors is substantially decreased or abolished, when the residual infection of cells containing such receptors with an adenovirus bearing such a modified fiber, is at least about one order of magnitude less than that observed with an adenovirus bearing a wild-type adenoviral fiber. Preferably, it is at least about two orders of magnitude, more preferably at least about three orders of magnitude, even more preferably at least about four orders of magnitude less than that observed with the corresponding wild-type adenovirus.

In one embodiment, the modified adenoviral fiber of the present invention is characterized in that it, comprises at least one mutation affecting one or more residues within the shaft and/or the knob, and especially within the AB loop, the CD loop, the DG loop and/or the beta sheet I of the knob. Of course, the modified fiber of the invention can combine several mutations which take place in one or more of the precited regions, e.g. in the knob AB loop and/or the CD loop and/or the DG loop and/or the beta sheet I.

Advantageously, the amino acid residue(s) to be mutated in the modified fiber of the invention is (are) within about 5 amino acids of an amino acid corresponding to residues 404-406, 449-454, 505-512, 551-560 of the wild-type Ad5 fiber (SEQ ID NO: 1). It is within the scope of those skilled in the art to identify the equivalent positions of these Ad5 fiber residues in another adenoviral fiber, oil the basis of available sequence database (see for example FIG. 9 of Xia et al., 1994, Structure 2, 1259-1270 giving alignment of the fiber knob regions of Ad2, Ad5, Ad3, Ad7, Ad40, Ad41 and CAV or Van Raaij, 1999, Virology 262(2), 333). More preferably, the mutation affects one or more amino acid residue(s) selected from the group of residues consisting of the threonine in position 404, the alanine in position 406, the valine in position 452, the lysine in position 506, the histidine in position 508, and the serine in position 555 of the wild type Ad5 fiber protein as shown in SEQ ID NO: 1. Even more preferably, the modified fiber protein of the invention comprises at least one substitution mutation of a residue corresponding to residues 404, 406, 452, 506, 508, and/or 555 of the wild-type Ad5 fiber (SEQ ID NO:1). Most preferably, said mutation of the Ad5 fiber comprises:
the substitution of the threonine in position 404 by a small aliphatic residue, such as alanine, proline or glycine, with a special preference for glycine,
the substitution of the alamine in position 406 by a basic residue such as lysine, arginine or histidine, with a special preference for lysine,
the substitution of the valine in position 452 by a basic residue such as lysine, arginine or histidine, with a special preference for lysine,
the substitution of the lysine in position 506 by a slightly basic amide residue such as glutamine or asparagine, with a special preference for glutamine,
the substitution of the histidine in position 508 by a basic residue such as lysine or arginine, with a special preference for lysine, or
the substitution of the serine in position 555 by a basic residue such as lysine, arginine or histidine, with a special preference for lysine,
Or any combination thereof.

As mentioned before, the present invention also encompasses a modified adenoviral fiber having more than one mutation. It could be advantageous to mutate two or more residues involved in the interaction with glycosaminoglycan (e.g. HSG receptors) and/or sialic acid-containing receptors, in order to further reduce or completely abolish its binding capability to one or both of these receptors. For example, the mutation of the glycine in position 450 by a lysine, of the threonine in position 451 by an asparagine and of the valine in position 452 by a lysine is advantageous in this respect. To further illustrate, mention can be made of the following examples of a fiber of Ad5, comprising:
the substitution of the lysine in position 506 by glutamine and the substitution of the histidine in position 508 by lysine (K506Q/H508K);
the substitution of the threonine in position 404 by glycine, the substitution of the lysine in position 506 by glutamine and the substitution of the histidine in position 508 by lysine (T404G/K506Q/H508K);
the substitution of the alanine in position 406 by lysine, the substitution of the lysine in position 506 by glutamine and the substitution of the histidine in position 508 by lysine (A406K/K506Q/H508K);
the substitution of the valine in position 452 by lysine, the substitution of the lysine in position 506 by glutamine and the substitution of the histidine in position 508 by lysine (V452K/K506Q/H508K);
the substitution of the lysine in position 506 by glutamine, the substitution of the histidine in position 508 by lysine and the substitution of the serine in position 555 by lysine (K506Q/H508K/S555K);

Other combinations such as T404G/A406K, T404G/V452K, T404G/K506Q, T404G/H508K, T404G/S555K, A406K/V452K, A406K/K506Q, A406K/H508K, A406K/S555K, A452K/K506Q, A452K/H1508K; A452K/S555K, K506Q/S555K, H508K/S555K, T404G/A406K/V452K, T404G/A406K/K506Q, T404G/A406K/H508K, T404G/A406K/S555K, T404G/V452K/K506Q, T404G/V452K/H508K, T404G/V452K/S555K, T404G/K506Q/S555K, T404G/A406K/V452K/K506Q/H508K/S555K etc. are also contemplated, by the present invention.

As another alternative, the modified adenoviral fiber of the invention originates (is obtained) from the wild type Ad2 fiber protein, and comprises at least one mutation of one or more amino acid residue(s) selected from the group of residues consisting of the threonine in position 404, the aspartic acid in position 406, the valine imposition 452, the lysine in position 506, the glutamine in position 508, and the threonine in position 556 of the wild type Ad2 fiber protein. Even more preferably, the modified fiber protein of the invention comprises at least one substitution mutation of a residue corresponding to residues 404, 406, 452, 506, 508, or 556 of the wild-type Ad2 fiber. Such substitutions of the Ad2 precited residues can be made by the type of residues as defined above for Ad5.

In accordance with the present invention, the modified adenoviral fiber of the present invention may further include at least one additional modification (e.g. amino acid substitution and/or deletion) other than those above-described. However, it is preferable not to drastically modify the three dimensional structure of the adenoviral fiber in order to preserve its trimerization properties and its function in the maturation of the corresponding viral particles. In this context, the amino acids forming a special structure (e.g. a bend) will be replaced with residues forming a similar structure, such as those mentioned in Xia et al. (1994). This make it possible to maintain the structure of the modified fiber of the invention, while at the same time confering upon it a property (e.g. host specificity) corresponding to that of the second adenovirus.

According to an advantageous embodiment, the modified adenoviral fiber of the present invention, further comprises at least one additional mutation affecting one or more amino acid residue(s) interacting with the CAR cellular receptor. In this regard and preferably, said modified adenoviral fiber has an affinity for said CAR cellular receptor and said glycosaminoglycan (e.g. HSG receptor) and/or sialic acid-containing cellular receptor of at least about one order of magnitude less than a wild-type adenoviral fiber, especially in the trimeric form. As before, the term [symbol] mutation [symbol] refers to deletion, addition or substitution or any combination thereof, with a special preference for substitution. Preferably the mutation aimed to abolish or reduce CAR binding affects one or more residue(s) located in the AB loop and/or the CD loop of the modified fiber of the invention.

As indicated in Xia et al. (1994), the host specificity of Ad2 and Ad5 is different from that of Ad3 and Ad7 with respect to CAR-mediated pathway. Thus, it would be advantageous to replace one or more residue(s) of a subgroup C (e.g. Ad5 or Ad2) fiber involved in CAR-binding with one or more residue(s) located in an equivalent position of a subgroup B (e.g. Ad3 or Ad7) fiber, so as to decrease the ability of said fiber to bind the CAR receptor. By way of illustration, suitable CAR-ablating mutations include those described in WO98/44121, WO01/6344, WO/0138361 and WO00/15823 as well as in Kirby et al. (2000, J. Virol. 74, 2804-2813) and Leissner et al. (2001, Gene Ther. 8, 49-57).

Preferably, the additional mutation (aimed to reduce or abolish CAR-binding) affects one or more residue(s) selected from the group consisting of the serine in position 408, the proline in position 409, the arginine in position 412, the lysine in position 417, the lysine in position 420, the tyrosine in position 477, the arginine in position 481, the leucine in position 485, the tyrosine in position 491, the alanine in position 494, the phenylalanine in position 497, the methionine in position 498, the proline in position 499 and the alanine in position 503 of the wild type Ad5 fiber protein (SEQ ID NO: 1). Even more preferably, the additional mutation is a substitution mutation of one or more residue corresponding to residues 408, 409, 412, 417, 420, 477, 481, 485, 491, 494, 497, 498, 499, or 503 of the wild type Ad5 fiber protein (SEQ ID NO: 1), and most preferably the additional mutation comprises:
the substitution of the serine in position 408 by glutamic acid (S408E),
the substitution of the proline in position 409 by lysine (P409K),
the substitution of the tyrosine in position 477 by alanine (Y477A),
the substitution of the leucine in position 485 by lysine (L485K),
the substitution of the tyrosine in position 491 by aspartic acid (Y491D),
the substitution of the alanine in position 494 by aspartic acid (A494D),
the substitution of the phenylalanine in position 497 by aspartic acid (F497D),
the substitution of the methionine in position 498 by aspartic acid (M498D),
the substitution of the proline in position 499 by glycine (P499G),
the substitution of the alanine in position 503 by aspartic acid (A503D), or
any combination thereof.

The modified adenoviral fiber of the invention can combine any mutation(s) affecting binding to native glycosaminoglycan (e.g. HGS receptors) and/or sialic acid-containing receptors and any additional mutation(s) affecting binding to CAR. Combination of the single S408E or A494D or A503D or the double A494D/A503D mutation affecting CAR binding and of the double K506Q/H508K or the triple T404G/K506Q/H508K, or A406K/K506Q/H508K or V452K/K506Q/H508K or K506Q/H508K/S556K mutation affecting HSG binding are suitable in the context of the present invention. Preferred examples include without limitation a modified adenoviral fiber comprising (i) the substitutiton of the serine in position 408 by glutamic acid, the substitutiton of the lysine in position 506 by glutamine and the substitutition of the histidine in position 508 by lysine (S408E/K506Q/H508K), (ii) the substitutiton of the alanine in position 503 by aspartic acid, the substitutiton of the lysine in position 506 by glutamine and the substitutiton of the histidine in position 508 by lysine (A503D/K506Q/H1508K), (iii) the substitutiton of the serine in position 408 by glutamic acid and the substitutiton of the serine in position 555 by lysine (S408E/S555K), or (iv) the substitutiton of the alanine in position 503 by aspartic acid and the substitutiton of the serine in position 555 by lysine (A503D/S555K).

The decrease or abolition of binding to CAR receptor provided by the modified fiber of the invention can be evaluated by infectivity or cell attachment as described above (e.g. cell attachment studies employing radiolabeled viruses or radiolabeled fibers recombinantly produced, Biacore techniques, immunochemistry, measurement of plaque formation, cytotoxicity, or gene delivery (e.g. using a reporter gene). It is also possible to probe a replica lift with radiolabeled CAR. Such techniques are described for example in WO01/16344 and WO01/38361 and Leissner et al. (2001, Gene Ther. 8, 49-57). For instance, infectivity can be studied in CAR+ cells (e.g. 293 cells or CHO cells transfected with a CAR-expressing plasmid) in the presence and in the absence of a competitor (i.e. soluble knob or anti-knob antibody). Infectivity or cell attachment of an adenovirus deficient or altered for CAR binding will not be substantially modified in the presence or in the absence of the competitor, whereas infectivity or cell attachment of a non modified (e.g. wild-type) adenovirus will be dramatically decrease in the presence of the competitor.

Advantageously, the ability of the modified fiber of the present invention to bind to the CAR is substantially decreased or abolished, when the residual infection of CAR+ cells measured with an adenovirus bearing such a modified fiber, is at least about one order of magnitude less than that observed with the wild type adenovirus. Preferably, it is at least about two orders of magnitude, more preferably at least about three orders of magnitude, even more preferably at least about four orders of magnitude less than that observed with the wild type adenovirus.

The modified adenoviral fiber of the invention can be further modified for example in the shaft region.

Preferably, the modified adenoviral fiber of the invention trimerizes when produced in an eukaryotic host cell.

The modified adenoviral fiber protein of the invention can be produced by any suitable method. For example, the modified adenoviral fiber can be synthetized using standard direct peptide synthesis techniques (e.g. as summarized in Bodanszky, 1984, Principle of Peptide Synthesis; Springer-Verlag, Heidelberg), such as via solid-phase synthesis (e.g. Merrifield, 1963, J. Am. Chem. Soc. 85, 2149-2154 and Barany et al., 1987, Int. J. Peptide Protein Res. 30, 705-739). Alternatively, oligonucleotide site-specific mutagenesis procedures are also appropriate to introduce the desired mutation(s) following cloning the sequence encoding a wild-type adenoviral fiber protein or peptide fragment into a vector (Bauer et al., 1985, Gene 37, 73 and Sculptor™ in vitro mutagenesis system, Amersham, Les Ullis France). Alternatively, site-specific mutation(s) can be introduced by PCR techniques. One engineered, the sequence encoding the modified adenoviral fiber protein or peptide fragment thereof can be subcloned into an appropriate vector using well known molecular techniques.

The present invention also relates to peptide fragment of the modified fiber protein of the invention. Within the context of the present invention, the term “peptide fragment” is intended to encompass peptide comprising at least a minimum of 6 consecutive amino acids of the modified fiber protein, preferably at least about 10, more preferably at least about 20, even more preferably at least about 40, and most preferably at least about 60, such consecutive amino acids bearing at least one of the mutation described herein. When such a peptide fragment is incorporated in place of an equivalent peptide fragment of a given wild-type adenoviral fiber, it confers a reduced affinity for a native glycosaminoglycan (e.g. HSG) and/or sialic acid-containing cellular receptor of at least about one order of magnitude less than said given wild-type adenoviral fiber, in particular in trimeric form.

The present invention also relates to a trimer comprising the modified adenoviral protein as defined above.

Any suitable assay can be employed to evaluate its ability to trimerize and/or associate with penton base. For example, the modified adenoviral fiber can be produced by standard recombinant techniques and these properties can be tested on the recombinant product. Any appropriate cloning or expression vector and corresponding suitable host cells can be used in the context of the present invention, including but not limited to bacteria (e.g. Escherichia coli), yeast, mammalian or insect host cell systems and established cell lines.

One assay for trimerization is evaluation of its solubility since it was shown that improperly folded monomers are generally insoluble (In Protein Purification, 3^{rd} Ed., 1994, Chap 9, p 270-282; Springer-Verlag, New york). Determination of the fiber solubility can be performed on radiolabelled recombinant fiber protein, following incorporation of radioactive amino acids into the protein during synthesis. Lysate from the host cell expressing the recombinant modified adenoviral fiber can be centrifuged and the supernatant and pellet can be assayed via a scintillation counter. Trimerization can also be evaluated by Western blot analysis (e.g. on SDS-PAGE gel) carried out on the supernatant and pellet obtained from cell lysate. Comparison of the amount of fiber protein detected from the pellet (insoluble) vis a vis the fiber protein detected from the supernatant (soluble) indicates whether the modified adenoviral fiber is soluble. Alternatively, trimerization can be assayed by using a monoclonal antibody recognizing only the trimeric form (e.g. via immunoprecipitation, Western blotting, etc.) (see for example Henry et al., 1994, J. Virol. 68, 5239-5246). Another evaluation of trimerization is the ability of the modified fiber to form a complex with the penton base (Novelli and Boulanger, 1995, Virol. 185, 1189), since only trimers can interact. This propensity can be assayed by co-immunoprecipitation, gel mobility-shift assays, SDS-PAGE, etc. Another measurement is to detect the difference in molecular weight of a trimer as opposed as a monomer. For example a boiled and denatured trimer will run as a lower molecular weight than a non-denatured stable trimer (Hong and Angler, 1996, J. Virol. 70, 7071-7078).

According to a preferred embodiment, the trimer according to the invention has an affinity for native glycosaminoglycan and/or sialic acid-containing receptors, and especially HSG receptors, of at least about one order of magnitude less than a wild type adenoviral fiber trimer. Methods for such measurement are indicated previously. Preferably, affinity for the trimer of the invention is at least about two orders of magnitude, more preferably at least about three orders of magnitude, even more preferably at least about four orders of magnitude less than that observed with the corresponding wild-type trimer.

According to another preferred embodiment, the trimer according to the invention containing a modified adenoviral fiber having additional mutation(s) as previously defined, further has an affinity for a native CAR cellular receptor of at least about one order of magnitude less than a wild type adenoviral fiber trifler. Reduction of CAR binding can be assayed as described above.

The present invention also relates to a DNA fragment or an expression vector encoding the modified fiber of the invention or a fragment thereof.

Within the context of the present invention, the term “DNA fragment” and “polynucleotide” are used interchangeably and define a polymeric form of any length of deoxyribonucleotides (DNA). The DNA fragment of the present invention can be linear or circular. It may also comprise modified nucleotides, such as methylated nucleotides or nucleotide analogs (see U.S. Pat. No. 5,525,711, U.S. Pat. No. 4,711,955 or EPA 302 175 as examples of modifications). If present, modifications to the nucleotide structure may be imparted before or after assembly of the polymer (such as by conjugation with a labeling component). The sequence of nucleotides may also be interrupted by non-nucleotide elements. The DNA fragment of the present invention can code for a full length modified fiber of an adenovirus serotype and also encompasses restriction endonuclease-generated and PCR-generated fragments that can be obtained therefrom. The present invention also encompasses synthetic fragments (e.g. produced by oligonucleotide synthesis).

Any type of vector can be used in the context of the present invention, whether of plasmid or viral, integrating or nonintegrating origin. Such vectors are commercially available or described in the literature. Similarly, those skilled in the art are capable of adjusting the regulatory elements required for the expression of the DNA fragment of the invention. Preferably, said vector is an adenoviral vector capable of producing under suitable culturing conditions, adenoviral particles bearing at their surface a modified fiber according to the present invention (as described hereinafter).

The present invention also relates to an adenoviral particle lacking a wild-type fiber and comprising the modified adenoviral fiber protein according to the present invention, and especially the trimer of the invention. The modified adenoviral fiber protein can be expressed from the adenoviral genome itself or provided in trans by a complementation cell line, such as one defined hereinafter. The adenoviral particle has a reduced capacity to interact with native glycosaminoglycan (e.g. HSG) and/or sialic acid-containing cellular receptors as compared to a wild-type particle, due to the above-mentioned reduction in affinity of the fibers present in said particle.

Moreover, the adenoviral particle of the invention can be further modified to exhibit reduced affinity for native cellular receptor(s) other than glycosaminoglycan (e.g. HSG) or sialic acid containing receptors, which are also involved in adenovirus attachment and/or entry into the permissive cells. In this context, the adenoviral particle of the invention can be further modified through the inclusion of additional mutation(s) in the modified fiber or in other viral protein(s) present at the surface of the particle. As discussed above, the adenoviral particle can include at least one additional mutation affecting one or more amino acid residue within a region of the adenoviral fiber interacting with the CAR cellular receptor, to also reduce its ability to interact with the CAR cellular receptor. Also the adenoviral particle of the invention can further comprise one or more penton base having a mutation affecting at least one native RGD sequence, preferably lacking a native RGD sequence, to reduce cell binding or entry via cellular integrins (see e.g. U.S. Pat. Nos. 5,559,099 and 5,731,190). But it has been observed that the integrin pathway is also inhibited with adenoviral particles exhibiting at their surface a trimer of modified fibers of the invention.

According to a preferred embodiment, the adenoviral particle of the invention, further comprises a ligand, for example, for targeting infection to a desired cell or cell population since the precited modification(s) alter(s) the native adenovirus tropism. Additionally, the ligand can be used to purify the virus, to inactivate the virus (e.g. by adsorbing it to a substrate for the ligand), or to grow the virus on cell lines having the receptors recognized by said ligand.

For the purpose of the present invention, the term [symbol] ligand [symbol] defines any entity capable of recognizing and binding, preferably with high affinity, an anti-ligand. It is evident by reading the specification that said ligand binds at least one cell-surface anti-ligand other than a native cellular receptor which normally mediates attachment and/or uptake of a wild type adenovirus (e.g. CAR, glycosaminoglycan (e.g. HSG) and/or sialic acid-containing cellular receptors). This anti-ligand can be expressed or exposed at the surface of a particular cell, the targeting of which is desired. It may be advantageous to target more particularly a tumor cell, an infected cell, a specific cell type or a category of cells. Therefore, suitable anti-ligands include without limitation polypeptides selected from the group consisting of cell-specific markers, tissue-specific receptors, cellular receptors, antigenic peptides (e.g. presented by histocompatibility antigens), tumor-associated markers, tumor-specific receptors, disease-specific antigens (e.g. viral antigens), and antigens specifically expressed on the surface of the target cells, etc. Such an anti-ligand can be naturally exposed at the surface of the targeted cell or subsequent to a modification of said target cell (e.g. upon treatment for example to reduce glycosylation or phosphorylation). The anti-ligand localized at the surface of a target cell is preferably one that a wild type adenoviral particle does not bind or binds but with a lower specificity than a adenoviral particle of the present invention. The binding specificity between a ligand and its corresponding anti-ligand can be determined according to techniques of the art, including ELISA, immunofluorescence and surface plasmon resonance-based technology (Biacore AB).

According to the invention, the ligand is localized on the surface of the claimed adenoviral particle in general, the ligand that may be used in the context of the present invention are widely described in the literature; it is a moiety able to confer to the adenoviral particle of the invention, the ability to bind to a given anti-ligand or a class of anti-ligands localized at the surface of at least one target cell.

In accordance with the aims pursued by the present invention, a ligand can be a lipid, a glycolipid, an hormone, a sugar, a polymer (e.g. PEG, polylysine, PEI, etc.), a polypeptide, an oligonucleotide, a vitamin, an antigen, a lectin, a polypeptide moiety presenting targeting property such as for example JTSI (WO94/40958), an antibody or combination thereof. The term [symbol] antibody [symbol] include but are not limited to monoclonal antibodies, antibody fragments (such as for example Fab and dAb antibody fragments), single chain antibodies (scFv) and a minimal recognition unit thereof (i.e. a fragment still presenting an antigenic specificity) such as those described in detail in immunology manuals (see for example Immunology, 3rd edition 1993, Roitt, Brostoff and Male, ed Gambli, Mosby). Suitable monoclonal antibodies to selected antigens may be prepared by known techniques, for example those disclosed in “Monoclonal Antibodies: A manual of techniques”, H. Zola (CRC Press, 1988) and in “Monoclonal Hybridoma Antibodies: Techniques and Applications”, J. G. R. Hurrell (CRC Press, 1982). Suitably prepared non-human antibodies may be “humanized” in known ways, for example by inserting the CDR regions of mouse antibodies into the framework of human antibodies. Additionally, as the variable heavy (VH) and variable light (VL) domains of the antibody are involved in antigen recognition, variable domains of rodent origin may be fused to constant domains of human origin such that the resultant antibody retains the antigenic specificity of the rodent parental antibody (Morrison et al (1984) Proc. Natl. Acad. Sci. USA 81, 6851-6855).

Alternatively, the ligand in use in the present invention can be derived from various types of combinatorial libraries, using well known strategies for identifying ligands (see U.S. Pat. No. 5,622,699). One approach uses recombinant bacteriophages to produce large libraries, as described in Scott and Smith, 1990, Science 249, 386-390; Cwirla et al., 1990, Proc. Natl. Acad. Sci. USA 87, 6378-6382; Devlin et al., 1990, Science 249, 404-406). A second approach uses primarily chemical methods, such as the Geysen method (Geysen et al., 1986, Molecular Immunology 23, 709-715; Geysen et al., 1987, J. Immunologic Method 102, 259-274) or the method of Fodor et al. (1991, Science 251, 767-773). Furka et al. (1991, Int. J. Peptide Protein res. 37, 487-493), U.S. Pat. No. 4,631,211 and U.S. Pat. No. 5,010,175 describe methods to produce a mixture of peptides that can be tested as targeting ligands. In another aspect, synthetic libraries (Needels et al., 1993, Proc. Natl. Acad. Sci. USA 90, 10700-10704; Ohlmeyer et al., 1993, Proc. Natl. Acad. Sci. USA 90, 10922-10926; WO92/00252 and WO94/28028) can be used to screen for targeting ligands.

Preferably, the ligand used in the present invention is a polypeptide having a minimal length of 6 amino acids. It is either a native polypeptide or a polypeptide derived from a native polypeptide. “Derived” means containing (i) one or more modifications with respect to the native sequence (e.g. addition, deletion and/or substitution of one or more residues), (ii) amino acid analogs, including not naturally occurring amino acids or (iii) substituted linkages as well as (vi) other modifications known in the art. The ligand can comprises sequences of various origins (e.g. a peptide that selectively bind a cell-surface anti-ligand fused to a protease recognition site) or sequence which are not contigous in the chain of amino acids in a given protein. In this context, it could be advantageous to use a ligand mimicking the particular conformation of a protein, e.g. in such a way to bring contigous and noncontigous sequences in mutual proximity. Preferably, the ligand does not comprise an oligomerization domain in order to not interfer with trimerization of the adenoviral fiber. In addition, the ligand may have a linear or cyclized structure (e.g. by flanking at both extremities a polypeptide ligand by cysteine residues). Additionally, the ligand moiety in use in the invention may include modifications of its original structure by way of substitution or addition of chemical moieties (e.g. glycosylation, alkylation, acetylation, amidation, phosphorylation, addition of sulfhydryl groups and the like). The invention further contemplates modifications that render the ligand detectable. For this purpose, modifications with a detectable moiety can be envisaged (i.e. a scintigraphic, radioactive, fluorescent, or dye labels and the like). Suitable radioactive labels include but are not limited to Tc^{99m}, I¹²³ and In¹¹¹. Such detectable labels may be attached to the ligand by any conventional techniques and may be used for diagnostic purposes (e.g. imaging of tumoral cells).

In one embodiment, the ligand allows to target a virally infected cell and is capable of recognizing and binding to a viral component (e.g. envelope glycoprotein, viral epitope) or capable of interfering with the virus biology (e.g. entry, replication . . . ). For example, the targeting of a HIV (Human Immunodeficiency Virus) infected cell can be performed with a ligand specific for an epitope of the HIV envelope, such as a ligand consisting of or derived from the 2F5 antibody (Buchacher et al., 1992, Vaccines 92, 191-195) recognizing a highly conserved epitope of the transmembrane glycoprotein gp41 or with a ligand moiety interferring with HIV attachment to its cellular receptor CD4 (e.g. the extracellular domain of the CD4 molecule). Suitable ligands also include those capable of recognizing and binding to cancer-associated viruses, such as human papilloma virus (HPV) associated with cervical cancer (e.g. by using a ligand directed to an HPV polypeptide including E6 and E7 early polypeptides as well as L1 and L2 late polypeptides), Epstein-Barr virus (EBV) associated with Burkitt's lymphomas (Evans et al., 1997, Gene Therapy 4, 264-267; e.g. by using a ligand directed to the EBV EBNA-1 antigen), polyoma virus, Hepatitis virus (e.g. by using a ligand directed to the E2 envelope polypeptide of the hepatitis C virus, Chan et al., 1996, J. Gen. Virol. 77, 2531). Such ligands are for example single chain antibodies recognizing one or more epitopes present in a viral envelope or core.

In another and preferred embodiment, the ligand allows to target a tumoral cell and is capable of recognizing and binding to a molecule related to the tumoral status, such as a tumor-specific antigen, a cellular protein differentially or over-expressed in tumoral cells or a gene product of a cancer-associated virus (as described above).

Examples of tumor-specific antigens include but are not limited to MUC-1 related to breast cancer (Hareuveni et al., 1990, Eur. J. Biochem 189, 475-486), the products encoded by the mutated BRCA1 and BRCA2 genes related to breast and ovarian cancers (Miki et al., 1994, Science 226, 66-71; Futreal et al., 1994, Science 226, 120-122; Wooster et al., 1995, Nature 378, 789-792), APC related to colon cancer (Polakis, 1995, Curr. Opin. Genet. Dev. 5, 66-71), prostate specific antigen (PSA) related to prostate cancer (Stamey et al., 1987, New England J. Med. 317, 909), carcinoma embryonic antigen (CEA) related to colon cancers (Schrewe et al., 1990, Mol. Cell. Biol. 10, 2738-2748), tyrosinase related to melanomas (Vile et al., 1993, Cancer Res. 53, 3860-3864), receptor for melanocyte-stimulating hormone (MSH) which is expressed in high number in melanoma cells, ErbB-2 related to breast and pancreas cancers (Harris et al., 1994, Gene Therapy 1, 170-175), and alpha-foetoprotein related to liver cancers (Kanai et al., 1997, Cancer Res. 57, 461-465). For example, a suitable ligand for targeting MUC-1 positive tumor cells can be a fragment of an antibody capable of recognizing and binding to the MUC-1 antigen, such as the scFv fragment of the SM3 monoclonal antibody which recognizes the tandem repeat region of the MUC-1 antigen (Burshell et al., 1987, Cancer Res. 47, 5476-5482 Girling et al., 1989, Int J. Cancer 43, 1072-1076; Dokurno et al., 1998, J. Mol. Biol. 284, 713-728).

Examples of cellular proteins differentially or overexpressed in tumor cells include but are not limited to the receptor for interleukin 2 (IL-2) overexpressed in some lymphoid tumors, for GRP (Gastrin Release Peptide) overexpressed in lung carcinoma cells, pancreas, prostate and stomach tumors (Michael et al., 1995, Gene Therapy 2, 660-668), TNF (Tumor Necrosis Factor) receptor, epidermal growth factor receptors, Fas receptor, CD40 receptor, CD30 receptor, CD27 receptor, OX-40, alphav integrins (Brooks et al., 1994, Science 264, 569) and receptors for certain angiogenic growth factors (Hanahan, 1997, Science 277, 48). Based on these indications, it is within the scope of those skilled in the art to define an appropriate ligand moiety capable of recognizing and binding to such proteins. To illustrate, IL-2 is a suitable ligand moiety to bind to IL-2 receptor.

In still another preferred embodiment, the ligand in use in the present invention allows to target tissue-specific molecules. A particular anti-ligand can be present on a narrow class of cell types or a broader group encompassing several cell types. The adenoviral particle of the invention can be targeted to cells within any organ or system, including for example, respiratory system (trachea, upper airways, lower airways, alveoly), nervous system and sensitory organs (e.g. skin, ear, nasal, tongue, eye), digestive system (e.g. oral epithelium, salivary glands, stomach, small intestines, duodenum, colon, gall bladder, pancreas, rectum), muscular system (e.g. cardiac muscle, skeletal muscle, smooth muscle, connective tissue, tendons, etc), immune system (e.g. bone marrow, stem cells, spleen, thymus, lymphatic system, etc), circulatory system (e.g. muscles connective tissue, endothelia of the arteries, veins, capillaries, etc), reproductive sytem (e.g. testis, prostate, cervix, ovaries), urinary system (e.g. bladder, kidney, urethra), endocrine or exocrine glands (e.g. breast, adrenal glands, pituitary glands), etc.

For example, ligands suitable for targeting liver cells include but are not limited to those derived from ApoB (apolipoprotein) able to bind to the LDL receptor, alpha-2-macroglobulin able to bind to the LPR receptor, alpha-1 acid glycoprotein able to bind to the asialoglycoprotein receptor and transferrin able to bind to the transferrin receptor. A ligand moiety for targeting activated endothelial cells may be derived from the sialyl-Lewis-X antigen (able to bind to ELAM-1), from VLA-4 (able to bind to the VCAM-1 receptor) or from LFA-1 (able to bind to the ICAM-1 receptor). A ligand derived from CD34 is useful to target the hematopoietic progenitor cells through binding to the CD34 receptor. A ligand derived from ICAM-1 is more intended to target lymphocytes through binding to the LFA-1 receptor. The targeting of T-helper cells may use a ligand derived from HIV gp-120 or a class II MHC antigen capable of binding to the CD4 receptor. The targeting of neuronal, glial, or endothelial cells can be performed through the use of ligands directed for example to tissue factor receptor (e.g. FLT-1, CD31, CD36, Cd34, CD105, CD13, ICAM-1; McCormick et al., 1998, J. Biol. Chem. 273, 26323-26329), thrombomodulin receptor (Lupus et al., 1998, Suppl. 2 S120, VEGFR-3 (Lymboussaki et al., 1998, Am. J. Pathol. 153, 395-403), VCAM-1 (Schwarzacher et al., 1996, Artherosclerosis 122, 59-67) or other receptors. The targeting of blood clots can be made via fibrinogen or aIIbb3 peptide. Finally, inflamed tissues can be targeted through selectins, VCAM-1, ICAM-1, etc.

Moreover, suitable ligands also include linear stretches of amino acids, such as polylysine, polyarginine and the like recognized by integrins. Also, a ligand can comprise a commonly employed tag peptide (e.g. short amino acids sequences known to be recognized by available antisera), such as sequences from glutathione-S-transferase (GST) from Shistosoma manosi, thioredoxin beta galactosidase, or maltose binding protein (MPB) from E. coli, human alkaline phosphatase, the FLAG octapeptide, hemagluttinin (HA).

It will be appreciated by those skilled in the art that ligand moieties which are polypeptides may be conveniently made using recombinant DNA techniques. The ligand moiety may be fused to a protein on the surface of the adenoviral particle of the invention or may be synthesized independently (e.g. by de novo synthesis or by expression of the encoding sequence in an eukaryotic or prokaryotic cell) and then coupled to the adenoviral particle as disclosed below. The nucleic acid sequences encoding many of the ligands encompassed by the present invention are known, for example those for peptide hormones, growth factors, cytokines and the like and may readily be found by reference to publically accessible nucleotide sequence databases such as EMBL and GenBank. Many cDNAs encoding peptide hormones, growth factors, all or part of antibodies, cytokines and the like, all of which may be useful as ligands, are generally commercially available. Once the nucleotide sequence is known it is obvious to the person skilled in the art how to make the sequence encoding the chosen ligand using, for example, chemical DNA synthetic techniques or by using the polymerase chain reaction to amplify the required DNA from genomic DNA or from tissue-specific cDNA.

Once a suitable ligand is identified, it can be incorporated into any location of the adenoviral particle of the invention, provided that it is still capable of interacting with its respective anti-ligand. In the context of the invention, said ligand is immunologically, chemically or genetically coupled to a viral polypeptide exposed at the surface of said adenoviral particle. Said viral polypeptide exposed at the surface of the adenoviral particle is selected from the group consisting of penton base, hexon, fiber, protein IX, protein VI and protein IIIa.

Chemical coupling of the selected ligand to the surface of the adenoviral particle may be performed directly through reactive functional groups (e.g. thiol or amine groups) or indirectly by a spacer group or other activating moiety. In particular, coupling may be done with (i) homobifunctional or (ii) heterobifunctional cross-linking reagents, with (iii) carbodiimides, (iv) by reductive amination or (vi) by activation of carboxylates (see for example Bioconjugate techniques 1996; ed G Hermanson; Academic Press).

Homobifunctional cross linkers including glutaraldehyde and bis-imidoester like DMS (dimethyl suberimidate) may be used to couple amine groups of the ligand to lipid structures containing diacyl amines. Many heterobifunctional cross linkers may be used in the present invention, in particular those having both amine reactive and sulfhydryl-reactive groups, carbonyl-reactive and sulfhydryl-reactive groups and sulfhydryl-reactive groups and photoreactive linkers. Suitable heterobifunctional crosslinkers are described in Bioconjugate techniques (1996) 229-285; ed. G Hermanson; Academic Press) and WO99/40214. Examples of the first category include but are not limited to SPDP (N-succinimidyl 3-(2-pyridyidithio)propionate), SMBP (succinimidyl-4-(p-maleimidophenyl)butyrate), SMPT (succinimidyloxycarbonyl-alpha-methyl-(alpha-2-pyridyidithio)toluene), MBS (m-maleimidobenzoyl-N-hydroxysuccinimide ester), SIAB (N-succinimidyl (4 iodoacetyl) aminobenzoate), GMBS (gamma-maleimidobutyryloxy) succinimide ester), SIAX (succinimidyl-6-iodoacetyl amino hexonate, SIAC (succinimidyl-4-iodoacetyl amino methyl), NPIA (p-nitrophenyl iodoacetate). The second category is useful to couple carbohydrate-containing molecules (e.g. env glycoproteins, antibodies) to sulfydryl-reactive groups. Examples include MPBH (4-(4-N maleimidophenyl)butyric acid hydrazide) and PDPH (4-(N-maleimidomethyl)cyclohexane-1-carboxyl-hydrazide (M₂C₂H and 3-2(2-pyridyldithio)proprionyl hydrazide). As an example of the third category, one may cite ASIB (1-(p azidosalicylamido)-4-(iodoacetamido)butyrate). Another alternative includes the thiol reactive reagents described in Frisch et al. (Bioconjugate Chem. 7 (1996) 180-186).

Chemical coupling between the ligand and the adenoviral particle of the invention may also be performed using a polymer such as polyethylene glycol (PEG) or its derivatives (see for example WO99/40214; Bioconjugate Techniques, 1996, 606-618; ed G Hermanson; Academic Press and Frisch et al., 1996, Bioconjugate Chem. 7, 180-186). The chemical coupling may also be non covalent, for example via electrostatic interactions (e.g. between a cationic ligand and a negatively charged adenoviral particle) or through the use of affinity components such as Protein A, biotin/avidin, which are able to associate both partners.

Immunological coupling involves antibodies to conjugate the selected ligand to the adenoviral particle of the invention For example, it is possible to use biotinylated antibodies directed to a surface-exposed viral epitope and streptavidin-labelled antibodies directed against the selected peptide ligand according to the technique disclosed by Roux et al. (1989, Proc. Natl. Acad Sci USA 86, 9079). Bifunctional antibodies directed against each of the coupling partners are also suitable for this purpose.

According to a preferred embodiment, the selected ligand is genetically coupled to the is adenoviral particle of the invention. Advantageously, the sequence encoding said ligand is inserted in the adenoviral genome, preferably within a gene encoding an adenoviral polypeptide localized at the surface. The present invention also encompass the use of specific signals (e.g. a membrane anchoring polypeptide) and peptide spacer (or linker) to further improve presentation of the ligand at the surface of the adenoviral particle. The term [symbol] peptide spacer [symbol] or [symbol] linker [symbol] as used herein refers to a peptide sequence of about one to 20 amino acids that is included to connect the ligand to the adenoviral polypeptide. The spacer is preferably made up of amino acid residues with high degrees of freedom of rotation, which permits the ligand to adopt a conformation that is recognized by its anti-ligand partner. Preferred amino acids for the spacer are alanine, glycine, proline and/or serine. In specific embodiments, the spacer is a peptide having the sequence Ser-Ala, Pro-Ser-Ala or Pro-Gly-Ser or a repetition thereof.

According to a first alternative, a portion of the surface-exposed adenoviral polypeptide can be removed and the ligand is inserted in replacement of the deleted portion. According to a second alternative, the ligand-encoding sequence is inserted in the viral sequence encoding the surface-exposed adenoviral polypeptide. Ligand insertion can be made at any location, at the N-terminus, the C-terminus or between two amino acid residues of the viral polypeptide. Preferably the insertion is made in frame and does not disrupt the viral open reading frame.

More preferably, the ligand is genetically coupled to a viral polypeptide exposed at the surface of the adenoviral particle of the invention, selected from the group consisting of penton base, hexon, fiber, protein IX, protein VI and protein IIIa at any suitable location. Where the ligand is inserted or replace a portion of the penton base, preferably it is within the hypervariable regions to ensure contact with the anti-ligand. Where the ligand is inserted or replace a portion of the hexon, preferably it is within the hypervariable regions. A suitable example is an adenovirus hexon comprising a deletion of about 13 amino acid residues from the HVR5 loop, corresponding to about amino acid residue 269 to about amino acid residue 281 of the Ad5 hexon and insertion of the ligand at the site of the deletion, eventually connected by a first spacer at the N-terminus and a second spacer at the C-terminus of the ligand. Even more preferably, the ligand is genetically inserted in the modified fiber of the invention, especially at the C-terminus or within the HI loop. More specifically, insertion in the HI loop may be made between about amino acid residue 538 to about amino acid residue 548 of the Ad5 fiber and insertion of the ligand at the site of the deletion, eventually connected by a first spacer at the N-terminus and a second spacer at the C-terminus of the ligand. Insertion at the C-terminus of the adenoviral fiber is generally made just upstream of the stop codon, optionally through the use of a peptide spacer connected at the N-terminus of the ligand. In a general manner, the insertion site is selected in such a way to maximally presentation of the ligand to the anti-ligand and to not disturb the interaction between the other viral proteins and fiber trimerization. Also, the ligand can be genetically inserted in the pIX protein, at any mocation but with a special preference for insertion at the C-terminus or within the C-terminal portion of pIX (e.g. in replacement of or in addition to one or more residues located within the 40 pIX residues preceeding the STOP codon). Where the ligand is inserted in the pIX protein, preferably pIX is also mutated in the coil coiled domain (as described for example in Rosa-Calavatra et al., 2001, J. Virol. 75, 7131-7141).

Of course, the adenoviral particle of the present invention can comprise more than one ligand, each binding to a different anti-ligand. For example, an adenoviral particle can comprise a first ligand permitting affinity-based purification and a second ligand that selectively bind a cell surface anti-ligand as described herein.

According to a particular case of the invention, the adenoviral particle of the invention is an [symbol] empty [symbol] capsid, i.e. it contains no nucleic acid. The use of such empty capsid is illustrated for example, for implementing DNA-based gene therapy protocols. In this respect, WO95/21259 describes a method for introducing a nucleic acid into a cell, using a combination of adenoviral particles and nucleic acids (e.g. naked nucleic acids). This method is based mainly on the capacity of the adenoviral particles to transport molecules to the cell nucleus after endocytosis. Curiel et al. (1992, Hum. Gene Ther. 3, 147-154) and Wagner et al; (1992, Proc. Natl. Acad. Sci. USA 89, 6099-6103) showed that complexation of plasmids with inactivated adenoviral particles allows the endosomes to be lysed before fusion with the lysosomes, therefore allowing the plasmids to escape degradation. This results in a 100- to 1000-fold increase in transfection efficiency in vitro.

According to a preferred embodiment, the adenoviral particle of the present invention comprises an adenoviral genome (reference will be also made to an adenoviral virus or adenoviral particle or adenovirus).

In one embodiment, the adenoviral genome is engineered to be conditionally replicative (CRAd adenovirus), in order to replicate selectively in specific cells (e.g. proliferative cells) as described for example in Heise and Kim (2000, J. Clin. Invest. 105, 847-851).

In another and preferred embodiment, the adenoviral genome is replication-defective, i.e. incapable of autonomous replication in the absence of complementation. The deficiency is obtained by a mutation or deletion of one or more viral gene(s) essential to the replication. It is preferably defective for at least the E1 function by total or partial deletion and/or mutation of one or more genes constituting the E1 region. Advantageously, the E1 deletion covers nucleotides (nt) 458 to 3328 or 458 to 3510 by reference to the sequence of the human adenovirus type 5 disclosed in the Genebank database under the accession number M 73260. Furthermore, the adenoviral backbone of the vector may comprise additional modifications (deletions, insertions or mutations in one or more other viral genes). An example of an E2 modification is illustrated by the thermosensible mutation affecting DBP (DNA Binding Protein) (Ensiniger et al., 1972, J. Virol. 10, 328-339). The adenoviral sequence may also be deleted of all or part of the E4 region. A partial deletion retaining the ORFs 3 and 4 or ORFs 3 and 6/7 may be advantageous (see for example European application EP 974 668; Christ et al., 2000, Human Gene Ther. 11, 415-427; Lusky et al., 1999, J. Virol. 73, 8308-8319). Additional deletions within the non-essential E3 region may increase the cloning capacity, however it may be advantageous to retain all or part of the E3 sequences coding for the polypeptides (e.g. gp19k) allowing to escape the host immune system (Gooding et al., 1990, Critical Review of Immunology 10, 53-71) or inflammatory reactions (EP 1203819). Second generation vectors retaining the ITRs and packaging sequences and containing substantial genetic modifications aimed to abolish the residual synthesis of the viral antigens may also be envisaged, in order to improve long-term expression of the expressed gene in the transduced cells (WO94/28152; Lusky et al., 1998, J. Virol 72, 2022-2032).

Adenoviruses adaptable for use in accordance with the present invention, can be derived from any human or animal source, in particular canine (e.g. CAV-1 or CAV-2; Genbank ref CAVIGENOM and CAV77082 respectively), avian (Genbank ref AAVEDSDNA), bovine (such as BAV3; Seshidhar Reddy et al., 1998, J. Virol. 72, 1394-1402), murine (Genbank ref ADRMUSMAV1), ovine, feline, porcine or simian adenovirus or alternatively from a hybrid thereof. Any serotype can be employed. However, the human adenoviruses of the C sub-group are preferred and especially adenoviruses 2 (Ad2) and 5 (Ad5). Generally speaking, the cited viruses are available in collections such as ATCC and have been the subject of numerous publications describing their sequence, organization and biology, allowing the artisan to apply them. It is preferred that the adenovirus be of the same subgroup or serotype that the adenovirus from which originates the modified fiber protein of the invention.

According to another preferred embodiment, the adenoviral particle of the invention is recombinant, i.e. the adenoviral genome comprises at least one gene of interest placed under the control of the regulatory elements allowing its expression in a host cell.

The term “gene of interest” refers to a nucleic acid which can be of any origin and isolated from a genomic DNA, a cDNA, or any DNA encoding a RNA, such as a genomic RNA, an mRNA, an antisense RNA, a ribosomal RNA, a ribozyme or a transfer RNA. The gene of interest can also be an oligonucleotide (i.e. a nucleic acid having a short size of less than 100 bp).

In a preferred embodiment, the gene of interest in use in the present invention, is a therapeutic gene, i.e. encodes a gene product of therapeutic interest. A “gene product of therapeutic interest” is one which has a therapeutic or protective activity when administered appropriately to a patient, especially a patient suffering from a disease or illness condition or who should be protected against a disease or condition. Such a therapeutic or protective activity can be correlated to a beneficial effect on the course of a symptom of said disease or said condition. It is within the reach of the man skilled in the art to select a gene encoding an appropriate gene product of therapeutic interest, depending on the disease or condition to be treated. In a general manner, his choice may be based on the results previously obtained, so that he can reasonably expect, without undue experimentation, i.e. other than practicing the invention as claimed, to obtain such therapeutic properties.

In the context of the invention, the gene of interest can be homologous or heterologous with respect to to the host cell or organism into which it is introduced. Advantageously, it encodes a polypeptide, a ribozyme or an antisense RNA. The term [symbol] polypeptide [symbol] is to be understood as any translational product of a polynucleotide whatever its size is, and includes polypeptides having as few as 7 amino acid residues (peptides), but more typically proteins. In addition, it may be of any origin (prokaryotes, lower or higher eukaryotes, plant, virus etc). It may be a native polypeptide, a variant, a chimeric polypeptide having no counterpart in nature or fragments thereof. Advantageously, the genie of interest in use in the present invention encodes at least one polypeptide that can compensate for one or more defective or deficient cellular proteins in an animal or a human organism, or that acts through toxic effects to limit or remove harmful cells from the body. A suitable polypeptide may also be immunity conferring and acts as an antigen, e.g. to provoke a humoral response.

Representative examples of polypeptides encoded by the gene of interest include without limitation polypeptides selected from the group consisting of:
polypeptides involved in the cellular cycle, such as p21, p16, the expression product of the retinoblastoma (Rb) gene, kinase inhibitors (preferably of the cycdin-dependent type), GAX, GAS-1, GAS-3, GAS-6, Gadd45 and cyclin A, B and D;
angiogenic polypeptides, such as members of the family of vascular endothelial growth factors (VEGF; i.e. heparin-binding VEGF Genbank accession number M32977), transforming growth factor (TGF, and especially TGFalpha and beta), epithelial growth factors (EGF), fibroblast growth factor (FGF and especially FGF alpha and beta), tumor necrosis factors (TNF, especially TNF alpha and beta), CCN (including CTGF, Cyr61, Nov. Elm-1, Cop-1 and Wisp-3), scatter factor/hepatocyte growth factor (SH/HGF), angiogenin, angiopoietin (especially 1 and 2), angiotensin-2, plasminogen activator (tPA) and urokinase (uPA);
cytokines, including interleukins (in particular IL-2, IL-6, IL-8, IL-12), colony stimulating factors (such as GM-CSF, G-CSF, M-CSF), interferons (such as IFN beta; Genbank accession number M25460; IFN gamma; Genbank accession number M29383 or IFN alpha);
chemokines, including RANTES, MIP alpha, MIP-1 beta, DCCK1, MDC, IL-10 (Genbank accession number U 16720) and MCP-1;
polypeptides capable of decreasing or inhibiting a cellular proliferation, including antibodies, toxins, immunotoxins, polypeptides inhibiting an oncogen expression products (e.g. ras, map kinase, tyrosine kinase receptors, growth factors), Fas ligand (Genbank accession number U08137), polypeptides activating the host immune system;
polypeptides capable of inhibiting a bacterial, parasitic or viral infection or its development, such as antigenic determinants, transdominant variants inhibiting the action of a viral native protein by competition (EP 614980, WO95/16780), immunoadhesin (Capon et al., 1989, Nature 337, 525-531; Bym et al., 1990, Nature 344, 667-670), immunotoxins (Kurachi et al., 1985, Biochemistry 24, 5494-5499) and antibodies (Buchacher et al., 1992, Vaccines 92, 191-195);
enzymes, such as urease, renin, thrombin, metalloproteinase, nitric oxide synthases (eNOS (Genbank accession number M95296) and iNOS), SOD, catalase, heme oxygenase (Genbank accession number X06985), enase, the lipoprotein lipase family; oxygen radical scavengers;
enzyme inhibitors, such as alpha1-antitrypsin, antithrombin III, plasminogen activator inhibitor PAI-1, tissue inhibitor of metalloproteinase 1-4;
polypeptides capable of restoring at least partially a deficient cellular function responsible for a pathological condition, such as dystrophin or minidystrophin (in the context of myopathies; England et al., 1990, Nature 343, 180-182), insulin (in the context of diabetes), hemophilic factors (for the treatment of hemophilias and blood disorders such as Factor VIIa (U.S. Pat. No. 4,784,950), Factor VIII (U.S. Pat. No. 4,965,199) or derivative thereof (U.S. Pat. No. 4,868,112 having the B domain deleted) and Factor IX (U.S. Pat. No. 4,994,371)), CFTR (in the context of cystic fibrosis; Riordan et al., 1989, Science 245, 1066-1072), erythropoïetin (anemia), lysosomal storage enzymes, including glucocerebrosidase (Gaucher's disease; U.S. Pat. No. 5,879,680 and U.S. Pat. No. 5,236,838), alpha-galactosidase (Fabry disease; U.S. Pat. No. 5,401,650), acid alpha-glucosidase (Pompe's disease; WO00/12740), alpha n-acetylgalactosaminidase (Schindler disease; U.S. Pat. No. 5,382,524), acid sphingomyelinase (Niemann-Pick disease; U.S. Pat. No. 5,686,240) and alpha-iduronidase (WO93/10244);
angiogenesis inhibitors, such as angiostatin, endostatin, platelet factor-4;
transcription factors, such as nuclear receptors comprising a DNA binding domain, a ligand binding domain and domain activating or inhibiting transcription (e.g. fusion products derived from oestrogen, steroid and progesterone receptors);
reporter genes (such as CAT, luciferase, eGFP . . . );
an antibody (whole immunoglobulins of any class, chimeric antibodies and hybrid antibodies with dual or multiple antigen or epitope specificities and fragments thereof such as F(ab)2, Fab′, Fab, scFv including hybrid fragments and anti-idiotypes) and
any polypeptides that are recognized in the art as being useful for the treatment or prevention of a clinical condition.

It is within the scope of the present invention that the gene of interest may include addition(s), deletion(s) and/or modification(s) of one or more nucleotide(s) with respect to the native sequence.

When the adenoviral particle of the present invention comprises a ligand aimed to target a tumor cell, the gene of interst preferably encodes an anti-tumor agent. A variety of anti-tumor agents may be utilized in accordance with the present invention. Within the context of the present invention, “anti-tumor agents” are understood to refer to compounds or molecules which inhibit the growth of a selected tumor. Representative examples of anti-tumor agents include immune activators and tumor proliferation inhibitors. Briefly, immune activators function by improving immune recognition of tumor-specific antigens (e.g. through humoral and/or cellular-mediated immunity). As a result, the immune system will more effectively inhibit or kill tumor cells. Immune activation may be subcategorized into immune modulators (molecules which affect the interaction between lymphocyte and tumor cell) and lymphokines, that act to proliferate, activate, or differentiate immune effector cells. Representative examples of immune modulators include CD3, ICAM-1, ICAM-2, LFA-1, LFA-3, beta-2-microglobulin, chaperones, alpha interferon and gamma interferon, B7/BB1 and major histocompatibility complex (MHC). Representative examples of lymphokines include gamma interferon, tumor necrosis factor, IL-1, IL-2, IL-3, IL4, IL-5, IL-6, IL-7, IL-8, IL-9, IL-10, IL-11, GM-CSF, CSF-1, and G-CSF.

Tumor proliferation inhibitors act by directly inhibiting cell growth, or by directly killing the tumor cell. Representative examples of tumor proliferation inhibitors include toxins and suicide genes. Representative examples of toxins include without limitation ricin (Lamb et al., 1985, Eur. J. Biochem. 148, 265-270), diphtheria toxin (Tweten et al., 1985, J. Biol. Chem. 260, 10392-10394), cholera toxin (Mekalanos et al., 1983, Nature 306, 551-557 Sanchez and Holmgren, 1989, Proc. Natl. Acad. Sci. USA 86, 481-485), gelonin (Stirpe et al., 1980, J. Biol. Chem. 255, 6947-6953), pokeweed (Irvin, 1983, Pharmac. Ther. 21, 371-387), antiviral protein (Barbieri et al., 1982, Biochem. J. 203, 55-59; Irvin et al., 1980, Arch. Biochem. Biophys. 200, 418-425; Irvin, 1975, Arch. Biochem Biophys. 169, 522-528), tritin, Shigella toxin (Calderwood et al., 1987, Proc. Natl. Acad. Sci. USA 84, 4364-4368; Jackson et al., 1987, Microb. Path. 2, 147-153) and Pseudomonas exotoxin A (Carroll and Collier, 1987, J. Biol. Chem. 262, 8707-8711).

[symbol] Suicide genes [symbol] can be defined in the context of the present invention as any gene encoding an expression product able to transform an inactive substance (prodrug) into a cytotoxic substance, thereby giving rise to cell death. The gene encoding the TK HSV-1 constitutes the prototype of the suicide gene family (Caruso et al., 1993, Proc. Natl. Acad. Sci. USA 90, 7024-7028; Culver et al., 1992, Science 256, 1550-1552). While the TK polypeptide is non toxic as such, it catalyzes the transformation of nucleoside analogs (prodrug) such as acyclovir or ganciclovir. The transformed nucleosides are incorporated into the DNA chains which are in the process of elongation, cause interruption of said elongation and therefore inhibition of cell division. A large number of suicide gene/prodrug combinations are currently available. Those which may more specifically be mentioned are rat cytochrome p450 and cyclophosphophamide (Wei et al., 1994, Human Gene Ther. 5, 969-978), Escherichia coli (E. coli) purine nucleoside phosphorylase and 6-methylpurine deoxyribonucleoside (Sorscher et al., 1994, Gene Therapy 1, 223-238), E. coli guanine phosphoribosyl transferase and 6-thioxanthine (Mzoz et al., 1993, Human Gene Ther. 4, 589-595). However, in a preferred embodiment, the adenoviral particle of the invention comprises a suicide gene encoding a polypeptide having a cytosine deaminase (CDase) or a uracil phosphoribosyl transferase (UPRTase) activity or both CDase and UPRTase activities, which can be used with the prodrug 5-fluorocytosine (5-FC). The use of a combination of suicide genes, e.g. encoding polypeptides having CDase and UPRTase activities, can also be envisaged in the context of the invention.

CDase and UPRTase activities have been demonstrated in prokaryotes and lower eukaryotes, but are not present in mammals. CDase is normally involved in the pyrimidine metabolic pathway by which exogenous cytosine is transformed into uracil by means of a hydrolytic deamination, whereas UPRTase transforms uracile in UMP. However, CDase also deaminates an analog of cytosine, 5-FC, thereby forming 5-fluorouracil (5-FU), which is highly cytotoxic when it is converted into 5-fluoro-UMP (5-FUMP) by UPRTase action.

Suitable CDase encoding genes include but are not limited to the Saccharomyces cerevisiae FCY1 gene (Erbs et al., 1997, Curr. Genet. 31, 1-6; WO93/01281) and the E. coli codA gene (EP 402 108). Suitable UPRTase encoding genes include but are not limited to those from E. coli (upp gene; Anderson et al., 1992, Eur. J. Biochem. 204, 51-56), Lactococcus lactis (Martinussen and Hammer, 1994, J. Bacteriol. 176, 6457-6463), Mycobacterium bovis (Kim et al. 1997, Biochem Mol. Biol. Int 41, 1117-1124), Bacillus subtilis (Martinussen et al. 1995, J. Bacteriol. 177, 271-274) and Saccharomyces cerevisiae FUR-1 gene; Kern et al., 1990, Gene 88, 149-157). Preferably, the CDase encoding gene is derived from the FCY1 gene and the UPRTase encoding gene is derived from the FUR-1 gene.

The present invention also encompasses the use of mutant suicide genes, modified by addition, deletion and/or substitution of one or several nucleotides providing that the cytotoxic activity of the gene product be preserved. A certain number of CDase and UPRTase mutants have been reported in the literature including a fusion protein which encodes a two domain enzyme possessing both CDase and UPRTase activities WO96/16183) as well as a mutant of the UPRTase encoded by the FUR-1 gene having the first 35 residues deleted (mutant FCU-1 disclosed in WO99/54481).

Additional examples of tumor proliferation inhibitors include antisense sequences which inhibit tumor cell growth by preventing the cellular synthesis of critical proteins needed for cell growth. Examples of such antisense sequences include antisense to positively-acting growth regulatory genes, such as oncogenes and protooncogenes (c-myc, c-fos, c-jun, c-myb, c-ras, Kc, JE, HER2), as well as antisense sequences which block any of the enzymes in the nucleotide biosynthetic pathway. Finally, tumor proliferation inhibitors also include tumor suppressors such as p53, retinoblastoma (Rb), and MCC and APC for colorectal carcinoma.

Sequences which encode the above-described anti-tumor agents may be obtained from a variety of sources. For example, plasmids that contain sequences which encode anti-tumor agents may be obtained from a depository such as the American Type Culture Collection (ATCC, Rockville, Md.), or from commercial sources such as British Bio-Technology Limited (Cowley, Oxford England). Alternatively, known cDNA sequences which encode anti-tumor agents may be obtained from cells which express or contain the sequences. Briefly, mRNA from a cell which expresses the gene of interest is reverse transcribed with reverse transcriptase using oligo dT or random primers. The single stranded cDNA may then be amplified by PCR utilizing oligonucleotide primers complementary to sequences on either side of desired sequences.

As mentioned above, the gene of interest is operably linked to regulatory elements allowing its expression in the host cell (e.g. the cell to be treated). Such regulatory elements include a promoter that may be obtained from any viral, bacterial or eukaryotic gene (even from the gene of interest) and be constitutive or regulable. Optionally, it can be modified in order to improve its transcriptional activity, delete negative sequences, modify its regulation, introduce appropriate restriction sites etc. Suitable promoters include but are not limited to the followings: adenoviral E1a, MLP, PGK, MT (metallothioneine; Mc Ivor et al., 1987, Mol. Cell Biol. 7, 838-848), alpha-1 antitrypsin, CFTR, surfactant, immunoglobulin, beta-actin, SRalpha, SV40, RSV LTR, TK-HSV-1, SM22, Desmin (WO 96/26284) and early CMV.

Preferably, the regulatory elements allowing the expression of the gene of interest are functional within a host cell presenting at its surface an anti-ligand to which the ligand in use in the invention binds. Said regulatory elements comprise a promoter preferably selected from the group consisiting of tissue-specific promoters and tumor-specific promoters. Suitable promoters include those functional in proliferative cells, such as those isolated from genes overexpressed in tumoral cells, such as the MUC-1 gene overexpressed in breast and prostate cancers (Chen et al., 1995, J. Clin. Invest. 96, 2775-2782), the CEA (Carcinoma Embryonic Antigen)-encoding gene overexpressed in colon cancers (Schrewe et al., 1990, Mol. Cell. Biol. 10, 2738-2748), the ERB-2 encoding gene overexpressed in breast and pancreas cancers (Harris et al., 1994, Gene Therapy 1, 170-175) and the alpha-foetoprotein-encoding gene overexpressed in liver cancers (Kanai et al., 1997, Cancer Res. 57, 461-465).

Those skilled in the art will appreciate that the regulatory elements controlling the expression of the gene of interest may further comprise additional elements for proper initiation, regulation and/or termination of transcription and translation of the gene(s) of interest into the host cell or organism. Such additional elements include but are not limited to non coding exon/intron sequences, transport sequences, secretion signal sequences, nuclear localization signal sequences, IRES, polyA transcription termination sequences, tripartite leader sequences, sequences involved in replication or integration. Said elements have been reported in the literature and can be readily obtained by those skilled in the art. Illustrative examples of introns suitable in the context of the invention include those isolated from the genes encoding alpha or beta globin (i.e. the second intron of the rabbit beta globin gene Green et al., 1988, Nucleic Acids Res. 16, 369; Karasuyama et al., 1988, Eur. J. Immunol. 18, 97-104), ovalbumin, apolipoprotein, immunoglobulin, factor IX, factor VII and CFTR and synthetic introns such as the intron present in the pCI vector (Promega Corp, pCI mammalian expression vector E1731) made of the human beta globin donor fused to the mouse immunoglobin acceptor or the intron 16S/19S of SV40 (Okayma and Berg, 1983, Mol. Cell. Biol. 3, 280-289). The additional elements may also contain a polyadenylation signal operably linked to the gene(s) of interest, to allow proper termination of the transcription. It is preferably positioned downstream of the gene of interest.

The gene of interest in use in the present invention can be inserted in any location of the adenoviral genome, with the exception of the cis-acting sequences (ITRs and packaging sequences). Preferably, it is inserted in replacement of a deleted region (E1, E3 and/or E4), with a special preference for the deleted E1 region. In addition, the expression cassette may be positioned in sense or antisense orientation relative to the transcriptional direction of the region in question.

The present invention encompasses the use of one or more gene(s) of interest. In this regard, the combination of genes encoding a suicide gene product and a cytokine (such as IL-2, IL-8, IFNgamma, GM-CSF) may be advantageous in the context of the invention. The different genes of interest may be controlled by common (polycistronic cassette) or independent regulatory sequences that are positioned either in the same or in opposite directions.

In addition, adenoviral particles or empty capsids of the invention can also be used to transfer nucleic acids (e.g. a plasmidic vector) by a viris-mediated cointernalization process as described in U.S. Pat. No. 5,928,944. This process can be accomplished in the presence of (a) cationic agent(s) such as polycarbenes or lipid vesicles comprising one or more lipid layers.

The adenoviral particle of the invention may be prepared and propagated according to any conventional technique in the field of the art (e.g. as described in Graham and Prevect, 1991, Methods in Molecular Biology, Vol 7, Gene Transfer and Expression Protocols; Ed E. J. Murray, The Human Press Inc, Clinton, N.J. or in WO96/17070)

The invention also relates to a process for producing the adenoviral particle according to the invention, comprising the steps of:
Introducing said adenoviral particle or the genome of said adenoviral particle into a suitable cell line,
Culturing said cell line under suitable conditions so as to allow the production of said adenoviral particle, and
Recovering the produced adenoviral particle from the culture of said cell line, and
Optionally purifying said recovered adenoviral particles.

The adenoviral particle or its genome is introduced into the cell in accordance with known techniques, such as transformation, transduction, microinjection of minute amounts of DNA into the nucleus of a cell (Capechi et al., 1980, Cell 22, 479-488), transfection for example with CaPO₄ (Chen and Okayama, 1987, Mol. Cell Biol. 1, 2745-2752), electroporation (Chu et al., 1987, Nucleic Acid Res. 15, 1311-1326), lipofection/liposome fusion (Felgner et al., 1987, Proc. Natl. Acad. Sci. USA 84, 7413-7417), particle bombardement (Yang et al., 1990, Proc. Natl. Acad. Sci. USA 87, 9568-9572), gene guns, infection (e.g. with an infective viral particle), direct DNA injection (Acsadi et al., 1991, Nature 352, 815-818), microprojectile bombardment (Williams et al., 1991, Proc. Natl. Acad. Sci. USA 88, 2726-2730), or the like.

With respect to cell line, both prokaryotic and eukaryotic cells may be employed, which include bacteria yeast, plants and animals, including human cells. Preferably, the adenoviral particle is replication-defective and said appropriate cell line complements at least one defective function of said adenoviral particle, eventually in combination with a helper virus. The cell lines 293 (Graham et al., 1977, J. Gen. Virol. 36, 59-72) and PERC6 (Fallaux et al., 1998, Human Gene Therapy 9, 1909-1917) are commonly used to complement the E1 function. Other cell lines have been engineered to complement doubly defective vectors (Yeh et al., 1996, J. Virol. 70, 559-565; Krougliak and Graham, 1995, Human Gene Ther. 6, 1575-1586; Wang et al., 1995, Gene Ther. 2, 775-783; Lusky et al., 1998, J. Virol. 72, 2022-2033; EP919627 and WO97/04119).

The present invention also encompasses a process for producing adenoviral particles lacking a functional fiber (by deleting all or part of the fiber-encoding sequence). In this case, the process of the invention employs preferably a cell line expressing a modified adenoviral fiber of the invention. Such a cell line comprises either in a form integrated into the genome or in episome form a DNA fragment or an expression vector of the present invention. Of course, the DNA fragment is placed under the control of appropriate translational and/or transcriptional regulatory elements to allow production of the modified adenoviral fiber of the invention in said cell line. Preferably, this cell line is further capable of complementing an one or more adenoviral functions selected from the group consisting of the functions encoded by the E1, E2, E4, L1, L2, L3, L4, L5 regions or any combination thereof. It is preferably produced from the 293 cell line or from the PER C6 cell line, e.g. by transfecting an expression vector encoding the modified fiber protein of the invention.

Alternatively, the process of the invention employs an adenoviral vector of the invention which genome contains the sequence encoding a modified fiber of the invention in replacement of the native fiber gene and two cell lines. First the adenoviral vector is introduced in a first cell line providing appropriate complementation according to the viral backbone (e.g. E1 for E1-deleted vectors) and further providing a wild-type fiber (e.g. 293 or PER-C6 transfected with an expression vector encoding the sequence encoding the corresponding wild-type fiber). This amplification step allows the recovery of adenoviral particles having a genome comprising the modified fiber-encoding sequence packaged in capsid having a wild-type fiber. The resulting adenovirus particles recovered from the culture of said first cell line are then used to infect a second cell line providing only the necessary complementation (e.g. 293 or PERC-6). Thus, after one round of amplification in such a second cell line, the produced adenoviral particles will be packaged in neosynthetized capsids comprising the modified fiber expressed from the adenoviral genome.

The adenoviral particles can be recovered from the culture supernatant but also from the cells after lysis and optionally further purified according to standard techniques (e.g. chromatography, ultracentrifugation, as described in WO96/27677, WO98/00524 WO98/26048 and WO00/50573).

The present invention also provides an eukaryotic host cell comprising the DNA fragment or the adenoviral particle of the present invention.

For the purpose of the invention, the term “host cells” should be understood broadly without any limitation concerning particular organization in tissue, organ, etc or isolated cells of a mammalian (preferably a human). Such cells may be unique type of cells or a group of different types of cells and encompass cultured cell lines, primary cells and proliferative cells from mammalian origin, with a special preference for human origin. Suitable host cells include but are not limited to hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g. skeletal muscle, cardiac muscle or smooth muscle), fibroblasts.

Moreover, according to a specific embodiment, the eukaryotic host cell of the invention can be further encapsulated. Cell encapsulation technology has been previously described (Tresco et al., 1992, ASAIO J. 38, 17-23; Aebischer et al., 1996, Human Gene Ther. 7, 851-860). According to said specific embodiment, transfected or infected host cells are encapsulated with compounds which form a microporous membrane and said encapsulated cells can further be implanted in vivo. Capsules containing the cells of interest may be prepared employing a hollow microporous membrane from poly-ether sulfone (PES) (Akzo Nobel Faser AG, Wuppertal, Germany; Deglon et al. 1996, Human Gene Ther. 7, 2135-2146). This membrane has a molecular weight cutoff greater than 1 MDa which permits the free passage of proteins and nutrients between the capsule interior and exterior, while preventing the contact of transplanted cells with host cells.

The present invention also relates to a composition comprising the host cell or the adenovirus particle of the invention, or which is produced using the process according to the invention, preferably a pharmaceutical composition, in combination with a vehicle which is acceptable from a pharmaceutical point of view. In a special case, the composition may comprise two or more adenoviral particles or eukaryotic host cells, which may differ by the nature (i) of the regulatory sequence and/or (ii) of the gene of interest and/or (iii) of the adenoviral backbone and/or (iv) the ligand.

The composition according to the invention may be manufactured in a conventional manner for a variety of modes of administration including systemic, topical and localized administration (e.g. topical, aerosol, instillation, oral). For systemic administration, injection is preferred, e.g. subcutaneous, intradermal, intramuscular, intravenous, intraperitoneal, intrathecal, intracardiac (such as transendocardial and pericardial), intratumoral, intravaginal, intrapulmonary, intranasal, intratracheal, intravascular, intraarterial, intracoronary or intracerebroventricular. Intramuscular, intravenous and intratumoral constitute the preferred modes of administration. The administration may take place in a single dose or a dose repeated one or several times after a certain time interval. The appropriate administration route and dosage may vary in accordance with various parameters, as for example, the condition or disease to be treated, the stage to which it has progressed, the need for prevention or therapy and/or the therapeutic gene to be transferred. As an indication, a composition based on adenoviral particles may be formulated in the form of doses of between 10⁴ and 10¹⁴ iu (infectious units), advantageously between 10⁵ and 10¹³ iu and preferably between 10⁶ and 10¹² iu. The titer may be determined by conventional techniques. The composition of the invention can be in various forms, e.g. in solid (e.g. powder, lyophilized form), liquid (e.g. aqueous).

Moreover, the composition of the present invention can further comprise a pharmaceutically acceptable carrier for delivering said adenoviral particle or eukaryotic host cell into a human or animal body. The carrier is preferably a pharmaceutically suitable injectable carrier or diluent which is non-toxic to a human or animal organism, at the dosage and concentration employed-(for examples, see Remington's Pharmaceutical Sciences, 16^{th} ed. 1980, Mack Publishing Co). It is preferably isotonic, hypotonic or weakly hypertonic and has a relatively low ionic strength, such as provided by a sucrose solution. Furthermore, it may contain any relevant solvents, aqueous or partly aqueous liquid carriers comprising sterile, pyrogen-free water, dispersion media, coatings, and equivalents, or diluents (e.g. Tris-HCl, acetate, phosphate), emulsifiers, solubilizers or adjuvants. The pH of the pharmaceutical preparation is suitably adjusted and buffered in order to be appropriate for use in humans or animals. Representative examples of carriers or diluents for an injectable, composition include water, isotonic saline solutions which are preferably buffered at a physiological or slightly basic pH (between about pH 8 to about pH 9, with a special preference for pH8.5). Suitable buffer include phosphate buffered saline, Tris buffered saline, mannitol, dextrose, glycerol containing or not polypeptides or proteins such as human serum albumin). A particularly preferred composition comprises an adenoviral particle in 1 M saccharose, 150 mM NaCl 1 mM MgCl₂, 54 mg/l Tween 80, 10 mM Tris pH 8.5. Another preferred composition is formulated in 10 mg/ml mannitol, 1 mg/ml HSA, 20 mM Tris, pH 7.2, and 150 mM NaCl. These compositions are stable at −70° C. for at least six months.

In addition, the composition according to the present invention may include one or more [symbol] stabilizing [symbol] additive(s), capable of preserving its degradation within the human or animal and/or of improving uptake into the host cell. Such additives may be used alone or in combination and include hyaluronidase (which is thought to destabilize the extra cellular matrix of the host cells as described in WO98/53853), chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile (see EP 890 362), cytokines, especially interleukin-10 (IL-10) (PCT/EP/99 03082), nuclease inhibitors such as actin G (WO99/56784) and cationic salts such as magnesium (Mg²⁺) (EP 998945) and lithium (Li⁺) (EP 99 40 3310.8) and any of their derivatives. The amount of cationic salt in the composition of the invention preferably ranges from about 0.1 mM to about 100 mM, and still more preferably from about 0.1 mM to about 10 mM. One may also employ substances susceptible to facilitate gene transfer in arterial cells, such as a gel complex of poly-lysine and lactose (Midoux et al., 1993, Nucleic Acid Res. 21, 871-878) or poloxamer 407 (Pastore, 1994, Circulation 90, 1-517).

The composition of the present invention is particularly intended for the preventive or curative treatment of disorders, conditions or diseases associated with cancer. The term “cancer” encompasses any cancerous conditions including diffuse or localized tumors, metastasis, cancerous polyps and preneoplastic lesions (e.g. dysplasies) as well as diseases which result from unwanted cell proliferation. A variety of tumors may be selected for treatment in accordance with the methods described herein. In general, solid tumors are preferred, although leukemias and lymphomas may also be treated especially if they have developed a solid mass, or if suitable tumor associated markers exist such that the tumor cells can be physically separated from nonpathogenic normal cells. For example, acute lymphocytic leukemia cells, may be sorted from other lymphocytes with the leukemia specific marker “CALLA”. Cancers which are contemplated in the context of the invention include without limitation glioblastoma, sarcoma, melanomas, mastocytoma, carcinomas (e.g. colorectal and renal cell carcinomas) as well as breast, prostate, testicular, ovarian, cervix (in particular, those induced by a papilloma virus), lung (e.g. lung carcinomas including large cell, small cell, squamous and adeno-carcinomas), kidney, bladder, liver, colon, rectum, pancreas, stomac, esophagus, larynx, brain, throat, skin, central nervous system, blood (lymphomas; leukemia, etc.), bone, etc cancers.

The composition of the invention may also be used for the prevention and treatment of other diseases, such as those affecting muscles, blood vessels (preferably arteries) and/or the cardiovascular system, including without limitation ischemic diseases (peripheral, lower limb, cardiac ischemia and angina pectoris), artherosclerosis, hypertension, atherogenesis, intimal hyperplasia, (re)restenosis following angioplasty or stent placement, neoplastic diseases (e.g. tumors and tumor metastasis), benign tumors, connective tissue disorders (e.g. rheumatoid arthritis), ocular angiogenic diseases (e.g. diabetic retinopathy, macular degeneration, corneal graft rejection, neovascular glaucoma), cardiovascular diseases (myocardial infarcts), cerebral vascular diseases, diabetes-associated diseases, immune disorders (e.g. chronic inflammation or autoimmunity), neurodegenerative diseases, Parkinson diseases and genetic diseases (muscular dystrophies such as Becker and Duchenne, hemophilias, Gaucher's disease, cystic fibrosis, etc. as listed above). Another application is to use the composition of the invention as in vivo expression system for disorders that involve the gene product to be secreted into the bloodstream, especially to restore protein deficiencies (e.g. hemophilia by expressing the appropriate coagulation factor, lysosomal storage diseases, anemias).

Moreover, in the composition of the invention, the adenoviral particle or the expression vector of the present invention may be conjugated to a lipid or polymer. In this respect, preferred lipids or polymers are cationic to interact with cell membranes (Felgner et al., 1989, Nature 337, 387-388). Cationic lipids or mixtures of cationic lipids which may be used in the present invention include Lipofectin™, DOTMA: N-[1-(2,3-dioleyloxyl)propyl]-N,N,N-trimethylammonium (Feigner, 1987, Proc. Natl. Acad. Sci. USA 84, 7413-7417), DOGS: dioctadecylamidoglycylspemine or Transfectam™ (Behr, 1989, Proc. Natl. Acad. Sci. USA 86, 6982-6986), DMRIE: 1,2-dimiristyloxypropyl-3-dimethyl-hydroxyethylammonium and DORIE: 1,2-diooleyloxypropyl-3-dimethyl-hydroxyethylammnoium (Felgner, 1993, Methods 5, 67-75), DC-CHOL: 3 [N—(N′,N′-dimethylaminoethane)-carbamoyl]cholesterol (Gao, 1991, BBRC 179, 280-285), DOTAP (McLachlan, 1995, Gene Therapy 2, 674-622), Lipofectamine™, spermine- and spermidine-cholesterol, Lipofectace™ (for a review see for example Legendre, 1996, Medecine/Science 12, 1334-1341 or Gao, 1995, Gene Therapy 2, 710-722) and the cationic lipids disclosed in patent applications WO 98/34910, WO 98/14439, WO 97/19675, WO 97/37966 and their isomers. Nevertheless, this list is not exhaustive and other cationic lipids well known in the art can be used in connection with the present invention as well. Cationic polymers or mixtures of cationic polymers which may be used in the present invention include chitosan (WO98/17693), poly(aminoacids) such as polylysine (U.S. Pat. No. 5,595,897 or FR 2 719 316); polyquaternary compounds; protamine; polyimines; polyethylene imine or polypropylene imine (WO 96/02655); polyvinylamines; polycationic polymer derivatized with DEAE, such as DEAE dextran (Lopata et al., 1984, Nucleic Acid Res. 12, 5707-5717); polyvinylpyridine; polymethacrylates; polyacrylates; polyoxethanes; polythiodiethylaminomethylethylene (P(TDAE)); polyhistidine; polyornithine; poly-p-aminostyrene; polyoxethanes; co-polymethacrylates (eg copolymer of HPMA; N-(2-hydroxypropyl)-methacrylamide); the compound disclosed in U.S. Pat. No. 3,910,862, polyvinylpyrrolid complexes of DEAE with methacrylate, dextran, acrylamide, polyimines, albumin, onedimethylami nomethylmethacrylates and polyvinylpyrrol idone-methylacrylaminopropyltrimethyl ammonium chlorides; polyamidoaminne (Haensler and Szoka, 1993, Bioconjugate Chem. 4, 372-379); telomeric compounds (patent application filing number EP 98401471.2); dendritic polymers (WO 95/24221). Nevertheless, this list is not exhaustive and other cationic polymers well known in the art can be used in the composition according to the invention as well. Colipids may be optionally included in order to facilitate entry of the vector into the cell. Such colipids can be neutral or zwitterionic lipids. Representative examples include phosphatidylethanolamine (PE), phospliatidylcholine, phosphocholine, dioleylphosphatidylethanolamine (DOPE), sphingomyelin, ceramide or cerebroside and any of their derivatives. The ratio of cationic lipids and/or cationic polymers to colipid(s) (on a weight to weight basis), when the co-lipid(s) is (are) co-existing in the complex, can range from 1:0 to 1:10. In preferred embodiments, this ratio ranges from 1:0.5 to 1:4.

The complexation of the adenoviral particle or expression vector of the invention with one or more of the above-cited compounds can be performed according to standard techniques. For example, the compound(s) (e.g. cationic lipids) is (are) dissolved in an appropriate organic solvent such as chloroform. The mixture is then dried under vaccum. The film obtained is further dissolved in an appropriate amount of solvent or mixture of solvents which are miscible in water, in particular ethanol, dimethylsulfoxide (DMSO), or preferably a 1:1 (v:v) ethanol:DMSO mixture, so as to form lipid aggregates according to a known method (WO 96/03977), or alternatively, is suspended in an appropriate quantity of a solution of detergent such as an octylglucoside (e.g. n-octyl-beta-D-glucopyranoside or 6-O—(N-heptylcarbamoyl)-methyl-alpha-D-glucopyranoside). The suspension may then be mixed with a solution comprising the desired amount of adenoviral particles. Subsequent dialysis may be carried out in order to remove the detergent and to recover the composition of the invention. The principle of such a method is described by Hofland et al. (1996, Proc. Natl. Acad. Sci. USA 93, 7305-7309).

The present invention also relates to the use of the expression vector, of the adenoviral particle or of the composition of the invention, or of an adenoviral particle which is produced using the process according to the invention, for the preparation of a drug intended for the treatment or the prevention of a disease in a human or animal organism by gene therapy.

Within the scope of the present invention, “gene therapy” has to be understood as a method for introducing any expressible sequence into a cell. Thus, it also includes immunotherapy that relates to the introduction of a potentially antigenic epitope into a cell to induce an immune response which can be cellular or humoral or both.

In a preferred embodiment, such a use is suitable for the treatment or the prevention of any of the diseases cited above, and more particularly cancer diseases. For this purpose, the adenoviral particle of the present invention may be delivered in vivo to the human or animal organism by specific delivery means adapted to this pathology. In this context, it is possible to operate via direct intratumoral injection. Alternatively, one may employ eukaryotic host cells that have been engineered ex vivo to contain the adenoviral particle according to the invention. Methods for introducing such elements into an eukaryotic cell are well known to those skilled in the art. The transfected/infected cells are grown in vitro and then reintroduced into the patient. The graft of encapsulated host cells is also possible in the context of the present invention (Lynch et al, 1992, Proc. Natl. Acad. Sci. USA 89, 1138-1142).

The present invention also relates to a method for the treatment of a human or animal organism, comprising administering to said organism a therapeutically effective amount of the adenoviral particle, the eukaryotic cell or the composition of the invention.

A [symbol] therapeutically effective amount [symbol] is a dose sufficient for the alleviation of one or more symptoms normally associated with the disease or condition desired to be treated. When prophylactic use is concerned, this term means a dose sufficient to prevent or to delay the establishment of a disease or condition.

The method of the present invention can be used for preventive purposes and for therapeutic applications relative to the diseases or conditions listed above. It is to be understood that the present method can be carried out by any of a variety of approaches. For this purpose, the adenoviral particle, the host cell or the composition of the invention can be is administered directly in vivo by any conventional and physiologically acceptable administration route, for example by intratumoral injection or by intravenous administration using specific delivery means adapted to this administration route. Alternatively, the ex vivo approach may also be adopted as described above to the invention into cells. Prevention or treatment of a disease or a condition can be carried out using the present method alone or, if desired, in conjunction with presently available methods (e.g. radiation, chemotherapy and/or surgery). For example, the method according the invention can be improved by combining injection with increase of permeability of a vessel. In a particular preferred embodiment, said increase is obtained by increasing hydrostatic pressure (i.e. by obstructing outflow and/or inflow), osmotic pressure (i.e. with hypertonic solution) and/or by introducing a biologically active molecule (i.e. histamine into the administered composition; WO98/58542). Furthermore, in order to improve the transfection rate, the patient may undergo a macrophage depletion treatment prior to administration of the composition of the invention (see for example Van Rooijen et al., 1997, TibTech, 15, 178-184).

As discussed above, the method of the present invention is more intended for the treatment of cancers, to provide tumor inhibition growth or tumor regression. For example, tumor inhibition may be determined by measuring the actual tumor size over a period of time. More specifically, a variety of radiologic imaging methods (e.g., single photon and positron emission computerized tomography; see generally, “Nuclear Medicine in Clinical Oncology,” Winkler, C. (ed.) Springer-Verlag, New York, 1986), may be utilized to estimate tumor size. Such methods may also utilize a variety of imaging agents, including for example, conventional imaging agents (e.g., Gallium-67 citrate), as well as specialized reagents for metabolite imaging, receptor imaging, or immunologic imaging (e.g., radiolabeled monoclonal antibody specific tumor markers). In addition, non-radioactive methods such as ultrasound (see, “Ultrasonic Differential Diagnosis of Tumors”, Kossoff and Fukuda, (eds.), Igaku-Shoin, New York, 1984), may also be utilized to estimate the size of a tumor.

In addition to the in vivo methods for determining tumor inhibition discussed above, a variety of in vivo methods may be utilized in order to predict in vivo tumor inhibition. Representative examples include lymphocyte mediated anti-tumor cytolytic activity determined for example, by a ⁵¹Cr release assay, tumor dependent lymphocyte proliferation (Ioannides et al., 1991, J. Immunol. 146, 1700-1707), in vitro generation of tumor specific antibodies (Herlyn et al., 1984, J. Immunol. Meth. 73, 157-167, cell (e.g., CTL, helper T cell) or humoral (e.g., antibody) mediated inhibition of cell growth in vitro (Gazit et al., 1992, Cancer Immunol. Immunother 35, 135-144), and, for any of these assays, determination of cell precursor frequency (Vose, 1982, Int. J. Cancer 30, 135-142).

Alternatively, inhibition of tumor growth may be determined based upon a change in the presence of a tumor marker. Examples include prostate specific antigen (“PSA”) for the detection of prostate cancer and Carcino-Embryonic Antigen (“CEA”) for the detection of colorectal and certain breast cancers. For yet other types of cancers such as leukemia, inhibition of tumor growth may be determined based upon the decreased numbers of leukemic cells in a representative blood cell count.

When the method of the invention uses recombinant adenoviral particle engineered to express a suicide gene, it can be advantageous to additionally administer a pharmaceutically acceptable quantity of a prodrug which is specific for the expressed suicide gene product. The two administrations can be made simultaneously or consecutively, but preferably the prodrug is administered after the adenoviral particle injection. By way of illustration, it is possible to use a dose of prodrug from 50 to 500 mg/kg/day, a dose of 200 mg/kg/day being preferred. The prodrug is administered in accordance with standard practice. The oral route is preferred. It is possible to administer a single dose of prodrug or doses which are repeated for a time sufficiently long to enable the toxic metabolite to be produced within the host organism or the target cell. As mentioned above, the prodrug ganciclovir or acyclovir can be used in combination with the TK HSV-1 gene product and 5-FC in combination with the cytosine deaminase and/or uracil phosphotransferase gene product.

Finally, the present invention also relates to the use of a modified adenoviral fiber, of a trimer therof, of an adenoviral particle, of a composition or of an eukaryotic host cell of the invention having the above-defined characteristics, to substantially reduce the binding to at least one native glycosaminoglycan and/or sialic acid-containing receptor, and especially to HSG receptors. Preferably, said modified adenoviral fiber, trimer therof, adenoviral particle, composition or eukaryotic host cell has an affinity for said native glycosaminoglycan and/or sialic acid-containing receptor of at least about one order of magnitude less as compared to a wild type adenoviral fiber, trimer therof, adenoviral particle, composition or eukaryotic host cell trimer.

In one embodiment, the modified adenoviral fibers, trimer therof, adenoviral particles, compositions or eukaryotic host cells of the invention are preferably used to substantially reduce or inhibit the binding to glycosaminoglycan-containing receptors, and especially to HSG receptors. In this regard, the amino acid residue(s) to be mutated in the modified fiber of the invention is (are) within about 5 amino acids of an amino acid corresponding to residues 404-406, 449-454, 505-512, 551-560 of the wild-type Ad5 fiber (SEQ ID NO: 1). It is within the scope of those skilled in the art to identify the equivalent positions of these Ad5 fiber residues in another adenoviral fiber, on the basis of available sequence database (see for example FIG. 9 of Xia et al., 1994, Structure 2, 1259-1270 giving alignment of the fiber knob regions of Ad2, Ad5, Ad3, Ad7, Ad40, Ad41 and CAV or Van Raaij, 1999, Virology 262(2), 333). More preferably, the mutation affects one or more amino acid residue(s) selected from the group of residues consisting of the threonine in position 404, the alanine in position 406, the valine in position 452, the lysine in position 506, the histidine in position 508, and the serine in position 555 of the wild type Ad5 fiber protein as shown in SEQ ID NO: 1. Even more preferably, the modified fiber protein of the invention comprises at least one substitution mutation of a residue corresponding to residues 404, 406, 452, 506, 508, and/or 555 of the wild-type Ad5 fiber (SEQ ID NO: 1). Most preferably, said mutation of the Ad5 fiber comprises:
the substitution of the threonine in position 404 by a small aliphatic residue, such as alanine, proline or glycine, with a special preference for glycine,
the substitution of the alanine in position 406 by a basic residue such as lysine, arginine or histidine, with a special preference for lysine,
the substitution of the valine in position 452 by a basic residue such as lysine, arginine or histidine, with a special preference for lysine,
the substitution of the lysine in position 506 by a slightly basic amide residue such as glutamine or asparagine, with a special preference for glutamine,
the substitution of the histidine in position 508 by a basic residue such as lysine or arginine, with a special preference for lysine, or
the substitution of the serine in position 555 by a basic residue such as lysine, arginine or histidine, with a special preference for lysine,
Or any combination thereof.

A representative example of such a combination includes the substitution of the lysine in position 506 by glutamine and the substitution of the histidine in position 508 by lysine (K506Q/H508K).

The reduction or alteration of the interaction of said modified fiber and the HSG receptors can be validated as described above and in Examples.

In a second embodiment, the modified adenoviral fibers, trimer therof, adenoviral particles, compositions or eukaryotic host cells of the invention are preferably used to substantially reduce or inhibit the binding to both glycosaminoglycan-containing receptors, and especially to HSG receptors, and CAR receptors. In this regard, the modified fiber combine any of the modification described above or any combination thereof with those described before in connection with CAR-ablated mutants. Preferred example include without limitation the use of a modified adenoviral fiber comprising (i) the substitutiton of the serine in position 408 by glutamic acid, the substitutiton of the lysine in position 506 by glutamine and the substitutiton of the histidine in position 508 by lysine (S408E/K506Q/H508K) (ii) the substitutiton of the alanine in position 503 by aspartic acid, the substitutiton of the lysine in position 506 by glutamine and the substitutiton of the histidine in position 508 by lysine (A503D/K506Q/1-1508K), (iii) the substitutiton of the serine in position 408 by glutamic acid and the substitutiton of the serine in position 555 by lysine (S408E/S555K), or (iv) the substitutiton of the alanine in position 503 by aspartic acid and the substitutiton of the serine in position 555 by lysine (A503D/S555K).

In a third embodiment, the modified adenoviral fibers, trimer therof, adenoviral particles, compositions or eukaryotic host cells of the invention are preferably used to substantially reduce or inhibit the binding to sialic acid-containing receptors. In this regard, the amino acid residue(s) to be mutated in the modified fiber of the invention is (are) within about 5 amino acids of an amino acid corresponding to residues 404-410 and 491-505 of the wild-type Ad5 fiber (SEQ ID NO: 1). It is within the scope of those skilled in the art to identify the equivalent positions of these Ad5 fiber residues in another adenoviral fiber, on the basis of available sequence database (see for example FIG. 9 of Xia et al., 1994, Structure 2, 1259-1270 giving alignment of the fiber knob regions of Ad2, Ad5, Ad3, Ad7, Ad40, Ad41 and CAV or Van Raaij, 1999, Virology 262(2), 333). Unexpectedly, it has been shown in this invention that adenovirus particles containing an Ad5 fiber modified in position 408 or 503 have a reduced infectivity toward a CAR⁻ lung cell line particularly rich ill sialic acids. In accordance with these data, a preferred use involves modified fibers or any element comprising such a fiber with mutation affecting one or more amino acid residue(s) selected from the group of residues consisting of the serine in position 408 and the alanine in position 503 and most preferably:
the substitution of the serine in position 408 by glutamic acid (S408E),
the substitution of the alanine in position 503 by aspartic acid (A503D), or
any combination thereof.

In accordance with the present invention, these modified fibers (e.g. in positions 408 and/or 503) and any elements containing such a fiber are preferably used to substantially reduce or inhibit the binding to both sialic acid-containing receptors and CAR receptors.

In a fourth embodiment, the modified adenoviral fibers, trimer therof, adenoviral particles, compositions or eukaryotic host cells of the invention are preferably used to substantially reduce or inhibit the binding to (i) glycosaminoglycan-containing receptors, and especially to HSG receptors, (ii) CAR receptors and (iii) sialic acid-containing receptors. In this regard, the modified fiber combine any of the modification described in connection with HSG-ablated mutants or any combination thereof with those described before in connection with CAR-ablated mutants and sialic-acid-ablated variant. Preferred examples include without limitation the use of a modified adenoviral fiber comprising (i) the substitutiton of the serine in position 408 by glutamic acid, the substitutiton of the lysine in position 506 by glutamine and the substitutiton of the histidine in position 508 by lysine (S408E/K506Q/H508K) (ii) the substitutiton of the alanine in position 503 by aspartic acid, the substitutiton of the lysine in position 506 by glutamine and the substitutiton of the histidine in position 508 by lysine (A503D/K506Q/H508K), (iii) the substitutiton of the serine in position 408 by glutamic acid and the substitutiton of the serine in position 555 by lysine (S408E/S555K), or (iv) the substitutiton of the alanine in position 503 by aspartic acid and the substitutiton of the serine in position 555 by lysine (A503D/S555K).

The use of such modified fibers according to the invention allows for reduction of the interaction of such a modified fiber with one or more specific cellular receptor(s) normally mediating adenovirus attachment and/or internalization into host cells, and thus significantly restrict the native tropism od such adenovirus particles. Incorporation of a specific targeting ligand as described above may be advantageous in this context, in order to redirect adenovirus infection to desired target cells.

The invention has been described in an illustrative manner, and it is to be understood that the terminology which has been used is intended to be in the nature of words of description rather than of limitation. Obviously, many modifications and variations of the present invention are possible in light of the above teachings. It is therefore to be understood that within the scope of the appended claims, the invention may be practiced in a different way from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### LEGENDS OF FIGURES

FIG. 1 illustrates the effect of soluble heparin on infection of CHO cells with a series of mutant adenoviruses having the indicated fiber mutations.

FIG. 2 illustrates competition assays performed on 293 cells infected with either wild-type (wt) or various adenovirus having the indicated fiber mutation(s) in the presence of 10 μg/ml of recombinant soluble knob.

FIG. 3 illustrates competition assays performed on CHO cells infected with either wild-type (wt) or various adenovirus having the indicated fiber mutation(s) pre-incubated with heparin (30 μg/ml, Sigma);

The following examples serve to illustrate the present invention.

### EXAMPLES

The constructs described below are prepared according to the general techniques of genetic engineering and of molecular cloning, detailed in Sambrook et al. (2001, Molecular Cloning; A Laboratory Manual, Cold Spring Harbor Laboratory Press, Cold Spring Harbor N.Y.) or according to the manufacturer's recommendations when a commercial kit is used. The cloning steps using bacterial plasmids are preferably carried out in the E. coli strain 5K (Hubacek and Glover, 1970, J. Mol. Biol. 50, 111-127) or in E. coli strain BJ5183 (Hanahan, 1983, J. Mol. Biol. 166, 557-580). The latter strain is preferably used for homologous recombination steps. The NM522 strain (Stratagene) is suitable for propagating the M13 phage vectors. The PCR amplification techniques are known to those skilled in the art (see for example PCR Protocols—A guide to methods and applications, 1990; Ed Innis, Gelfand, Sninsky and White, Academic Press Inc). With respect to the repair of restriction sites, the technique used consists in filling the overhanging 5′ ends using the large fragment of E. coli DNA polymerase 1 (Klenow). The Ad5 nucleotide sequences are those disclosed in the Genebank database, under the reference M73260.

With regard to the cell biology, the cells are transfected according to standard techniques known to those skilled in the art. Mention may be made of the calcium phosphate precipitation technique, but any other protocol can also be used, such as the DEAE dextran technique, electroporation, methods based on osmotic shocks, or methods based on the use of cationic lipids. In the examples which follow, use is made of the human embryonic kidney 293 cell line (ATCC CRL1573), the CHO cell line (ATCC; CCL-61) and 293-Fiber cells (293-Fb), which constitutively express the adenovirus type 5 fiber protein (described previously in Legrand et al., 1999, J. Virol. 73, 907-919). The culturing conditions are conventional in the art. For illustrative purposes, the cells are grown at 37° C. in DMEM (Gibco) supplemented with 10% Fetal Calf Serum and antibiotics.

Materials and Methods

Construction of Fiber-Modified Viral Genomes

All cloning steps were performed using standard molecular biology techniques. In order to introduce mutations in Ad5 fiber knob, a mutagenesis template for the Sculptor™ in vitro mutagenesis system (Amersham, Les Ulis, France) was first generated. The template single-strand DNA m13F5knob contains Ad5 sequence from nucleotide 31994 (HindIII site) to nt 32991 (SmaI site) (Santis et al., 1999, J. Gen. Virol. 80, 1519-1527) The substitution mutations were introduced with the following antisense oligonucleotides:

| | |
|---|---|
| CAR minus | |
| Ser408Glu: | |
| 5′- gc att tag tct aca gtt agg ctc tgg agc tgg tgt ggt cca c-3′; | (OTG12499; SEQ ID NO: 2) |
| Ala494Asp (A494D): | |
| 5′- gttaggcataaatccaacgtcgtttgtataggctgtgcc-3′; | (OTG12728; SEQ ID NO: 3) |
| Ala503Asp (A503D): | |
| 5′- accgtgagattttggatagtctgataggttaggcataaa-3′; | (OTG12737; SEQ ID NO: 4) |
| Heparan minus | |
| Thr404Gly (T404G): | |
| 5′-ctacagttaggagatggagcgggcccggtccacaaagttagcttatc-3′ | (OTG12740; SEQ ID NO: 5) |
| Ala406Lys (A406K): | |
| 5′- gtctacagttaggagatggctttggtgtggtccacaaag-3′; | (OTG12498; SEQ ID NO: 6) |
| Val452Lys (V452K): | |
| 5′- aagatgagcactttgctttgttccagatattgg-3′; | (OTG12500; SEQ ID NO: 7) |
| Ser555Lys (S555K): | |
| 5′- gtggccagaccagtcccacttaaatgacatagagtatgc-3′; | (OTG12506; SEQ ID NO: 8) |

The double mutation Lys506Q/His508Lys (K506QH508K) was introduced with following antisense oligonucleotides: 5′-acttttggcagttttacccttagactgtggataagctgataggtt-3′ (OTG12738; SEQ ID NO:9).

Ad vectors deficient for CAR and Heparan sulfate proteoglycan pathways were constructed with combination of the single S408E or A494D or A503D or the double A494D/A503D CAR mutations and above triple heparan sulfate mutations K506QH508K/T404G, or K506QH508K/A406K, or K506QH508K/V452K, or K506QH508K/S555K.

The modified fiber was further modified by incorporation of a ligand (7 lysine residues also designated 7K) and a flexible linker at the C-terminal extremity of the fiber. For this purpose, the single strand template (either wild type (wt) or mutated m13F5knob) was mutated using oligonucleotide OTG7000 (5′-aac gat tct tta gct gcc ggg age aga ggc gga ggc gga ggc gct ggg ttc ttg ggc aat-3′ SEQ ID NO: 10) in order to introduce a 12 amino acid linker (ProSerAlaSerAlaSerAlaSerAlaProGlySer) and then with OTG12125 (5′-cac aaa cga tct tta ctt ctt ctt ttt tct tct ttt tgg atc egg gag cga ggc gga g-3′ SEQ ID NO: 11) to add the 7 lysine residues.

The HindIII-SmaI fragments isolated from the mutated m13F5knob plasmids were directly introduced by homologous recombination into the BstBI-restricted pTG4213. This plasmid contains a β-galactosidase expressing E1-deleted Ad5 genome in which a unique BstBI site was introduced at nucleotide 32940, downstream of the fiber stop codon. The generation of the pTG4213 was as follows: m13F5knob was mutated with OTG7213 (5′-t gaa aaa tga ttc gaa att ttc tgc a-3′ SEQ ID NO: 12) to introduce an unique BsiBI site (sequence in bold). The isolated HindIII-SmaI fragment was cloned by homologous recombination in the E. coli BJ5183 in pTG8533, a transfer plasmid bearing an Ad5 segment extending from nt 21562 to the right-end ITR. Thereafter, the purified BstEII fragment (nt 24843-35233) was introduced into the Ad5 genome by homologous recombination with pTG3602, a plasmid containing the full length Ad5 genome (described in Chartier et al., 1996, J. Virol. 70, 4805-4810). The replacement in this backbone of the E1 region with the MLP driven-βgalactosidase expression cassette was performed as described previously (Legrand et al., 1999, J. Virol. 73, 907-919).

Virus, Production and Titration

Five μg of the fiber-modified viral genomes were excised from the plasmid backbone by PacI digestion and transfected into 293 or 293-Fb cells. Cells were then recovered 2 weeks post-transfection for further analysis and expansion either in wild type 293 cells or in 293-Fb cells, depending on the required complementation (Legand et al., 1999, J. Virol. 73, 907-919). Primary viral stocks were then amplified oil 293 cells. Virus purification, titration and storage were as described (Lusky et al., 1999, J. Virol. 73, 8308). Virus particle concentration (P/ml) was measured by optical density (of one OD₂₆₀ corresponds to 1.1×10¹² particles/ml). Infectious titers (Infection Unit (IU)/ml) were determined 16 h to 20 h post-infection of 293 cells by staining for β-galactosidase activity (Janes et al., 1986, EMBO J. 5, 3133). The integrity of the viral genome and the presence of the fiber mutation were verified by analysing viral DNA, extracted using the Hirt method (Gluzinan et al., 1983, J. Virol. 45, 91).

Analysis of the Adenoviral Protein Profile

2×10¹⁰ purified viral particles were diluted in 2× Laemmli buffer, incubated for 5 min at 95° C. and loaded onto a 10% SDS-polyacrylamide gel. The proteins were detected by silver staining (Wray et al., 1981, Anal. Biochem. 118, 197). Specific detection of the fiber or penton base proteins was performed as previously described (Legrand et al., 1999, J. Virol. 73, 907-919).

Competition Experiments

As competitor for CAR entry process, purified Ad5 knob (10 μg/ml) were used. Target cell monolayer were incubated for one hour at 4° C. with either PBS or knob molecules. Ad-LacZ bearing either a wild-type or a codified fiber, diluted with 2% FCS-containing DMEM medium, were then added to 293 cells for one hour. Cells were then incubated at 37° C. for 24 or 48 h pi. After incubation for 24 or 48 hours at 37° C., cells were fixed and stained for beta-galatosidase activity. Alternatively, the beta-galatosidase activity of whole cell lysate was monitored using chemiluminescent substrate (luminescent beta-galatosidase detection kit; Clontech, Palo Alto, Calif., USA).

The same technique can be used to evaluate integrin-mediated entry process of the different modified fibers, with the exception that a RGD peptide (4 mg/ml, Neosystem, Strasbourg) was used as a competitor.

As competitor of HSG entry pathway, heparin (3 mg/ml, Sigma, St quentin, France) was used as previously described (Dechecchi et al., 2001, J. Virol. 75, 8772-8780). Wild-type and mutated Ad-LacZ adenoviruses were preincubated for one hour at 37° C. with heparin (Sigma) at a concentration of 30 μg/ml in 20 μl of 2% FCS-containing DMEM medium. The pretreated Adenovirus suspension was then diluted with ice-cold 2% FCS-containing DMEM medium at wanted concentration and added to CHO cells (CAR−) for one hour incubation on ice. Cells were then incubated at 37° C. for 24 or 48 h pi. Cells were fixed and stained for beta-galatosidase activity. Alternatively, the beta-galatosidase activity of whole cell lysate was monitored using chemiluminescent substrate (luminescent beta-galatosidase detection kit; Clontech, Palo Alto, Calif., USA).

Inhibition of adenovirus binding to HSG receptors was performed following heparinase treatment. For this purpose, target cell monolayer were incubated for one hour at 37° C. with a mix of Heparin Lyase I, II, III (Sigma) at concentration of 100 U/ml. Wild-type or modified Ad-LacZ, diluted with ice-cold 2% FCS-containing DMEM medium at wanted concentration, were then added to CHO cells for one hour incubation on ice. Cells were then incubated at 37° C. for 24 or 48 h pi. After incubation for 24 or 48 hours at 37° C., cells were fixed and stained for beta-galatosidase activity. Alternatively, the beta-galatosidase activity of whole cell lysate was monitored using chemiluminescent substrate (luminescent beta-galatosidase detection kit; Clontech, Palo Alto, Calif., USA).

### Example 1

### Construction of Fiber Mutants Impaired in the HSG Entry Pathway and Properties of the HSG-Mutant Viruses

1.1 Incorporation of Modified Fiber in Purified Adenoviral Particles.

Mutations of the Ad5 fiber gene and corresponding adenoviral particles were generated as described in the Materials and Methods. We sought to investigate the fiber residues involved in binding to the HSG receptors which have been recently described as cellular receptors for adenovirus inepenidently of CAR. A summary of the fiber mutations altering binding to HSG receptors is provided in Table 2.

TABLE 2
Description of the liber Heparan-mutations

| Location in the knob | Mutations performed |
|---|---|
| AB loop (aa 403-418) | Thr404Gly |
| | Ala406Lys |
| CD loop (aa 441-453) | Val452Lys |
| DG loop (aa 462-514) | Lys506Gln/His508Lys |
| I sheet (aa 550-557) | Ser555Lys |

We first evaluated the effect of these mutation on adenoviral capsid formation. The various fiber-modified viruses produced on 293 cells were purified by cesium chloride gradient (density 1.34 g/ml). 2×10¹⁰ purified particles were subjected to a 4-12% Bis-Tris Nupage gel and transferred to nitrocellulose. Filters were hybridized with either an anti-penton base serum or an anti-fiber antibody and were then treated with a secondary horseradish peroxidase-conjugated donkey anti-rabbit antibody.

The incorporation of the modified fiber into de viral particles was studied by Western blot analysis using sera directed against the Ad5 Knob (provided by Dr. Gerard; Henry et al., 1994, J. Virol. 68, 5239-5246) and the penton base (a polyclonal rabbit anti-penton antibody provided by Pr. Boulanger), as control. A strong positive signal was observed for the wt Ad-LacZ virus and all the mutated fiber vectors at the expected molecular weight.

These results demonstrate that the pre-cited mutations have no deleterious effect on the correct folding of the fiber protein and do not prevent its assembly into the capsid.

1.2 Maturation of Fiber-Modified Viruses

The protein profile of the mutant adenovirus particles bearing a modified fiber as described above was analyzed and compared to Ad-LacZ (having the Ad5 wild-type fiber) and a fiber-deleted controls (Ad-LacZ/Fb^{o}). For this purpose, the various fiber-modified viruses and controls (AdLacZ and Ad-lacZ/Fb^{o}) were produced oil 293 cells and purified on cesium chloride gradient. 2×10¹⁰ purified particles were subjected to a 10% SDS-polyacrylamide gel subsequently revealed by silver staining.

It was found that the majority of the fiber-modified viruses exhibit the same protein profile as the Ad-LacZ. With the exception of the mutant virus bearing the A406K fiber, the modified fiber proteins are present in the viral particle in stoichiometric amounts as the wild-type adenovirus. On marked contrast, the fiber-deleted Ad-LacZ/Fb^{o} virus still contains precursors of hexon-associated protein (pVI), of minor core protein (pVII) and of pVIII protein, indicative of an incomplete proteolytic processing.

1.3 Growth Characteristics of Fiber-Modified Viruses on 293 Cells

Growth properties of the precited fiber-modified Ad were analysed on 293 cells. For this purpose, 293 cells were infected at an MOI of 1 IU/cell with the control viruses Ad-LacZ or Ad-LacZ/Fb^{o} or with the various fiber-modified viruses. Infected cells and supernatant were harvested at 24, 48, 56, 64 and 72 h post-infection and were treated by three freeze-thawing cycles to release virus particles. Titers of released viruses were determined by beta-galactosidase staining.

As a result, the propagation of the fiber-modified viruses is not significantly altered as compared to the wild-type adenovirus Ad-LacZ. Consistent with this observation, the titers of infectious mutant virus (IU/ml) after large-scale production was not markedly reduced compared to the titer obtained with adenovirus bearing a wild type fiber, as well as the p/IU ratio. This is the consequence of the ability of the mutant adenovirus bearing HSG-ablated fiber to entry 293 cells via CAR receptor. In marked contrast, propagation of CAR-ablated viruses (as described in Leissner et al., 2001, Gene Ther. 8, 49-57) is greatly altered in 293 cells as well as that of fiber deleted mutants Ad-LacZ/Fb^{o}, as evidenced by the poor formation of infectious units (large augmentation of the IU/perticle ratio).

A summary of these results is provided in Table 3

TABLE 3
Physical characteristics of the fiber mutant viruses

| | | Infectious Unit/ml | |
|---|---|---|---|
| virus | Particle/ml | (293 cells) | IU/Particle |
| Ad-LacZ | 2.5 × 10¹² | 1.3 × 10¹¹ | 1/40 |
| Ad-LacZ/Fb° | 4.9 × 10¹² | 1.1 × 10⁷ | 1/400000 |
| Ad-LacZ/Fb-Thr404gly | 3.1 × 10¹² | 2.6 × 10⁸ | 1/12000 |
| Ad-LacZ/Fb-Ala406Lys | 2.2 × 10¹² | 2.9 × 10⁸ | 1/8000 |
| Ad-LacZ/Fb-Val452Lys | 8.8 × 10¹¹ | 1.8 × 10⁹ | 1/500 |
| Ad-LacZ/Fb- | 1.2 × 10¹² | 9.1 × 10⁸ | 1/1300 |
| Lys506Q/His508Lys | | | |
| Ad-LacZ/Fb-Ser555Lys | 1.4 × 10¹² | 4 × 10¹⁰ | 1/40 |

1.4 Ability of Fiber-Modified Ad Virus to Infect CHO Cells in the Presence of Saturating Concentration of Soluble Heparin.

A series of fiber-mutated adenoviruses (including those of Table 2) were evaluated for their ability to bind cellular HSG receptors by a competition assay using soluble heparin. One hundred IU/10⁵ cells of Ad-LacZ (equiped with a wild type fiber; corresponding to 4×10³ Particles (P)/10⁵ cells), fiberless Ad-LacZ/Fb^{o} (corresponding to 4×10⁷P/10⁵ cells) or a series of fiber-modified Ad expressing LacZ. (corresponding to 5×10⁴ to 3×10⁷P/10⁵ cells) were pre-incubated with heparin (30 μg/ml, Sigma) and then added to CHO cells. 48 h post-infection, the cells were stained for β-galactosidase expression. The efficiency of infection was expressed as the percentage of β-galactosidase positive cells in the absence of heparin. The number of blue cells counted in the control wells (in the absence of knob) ranges from 100 to 400.

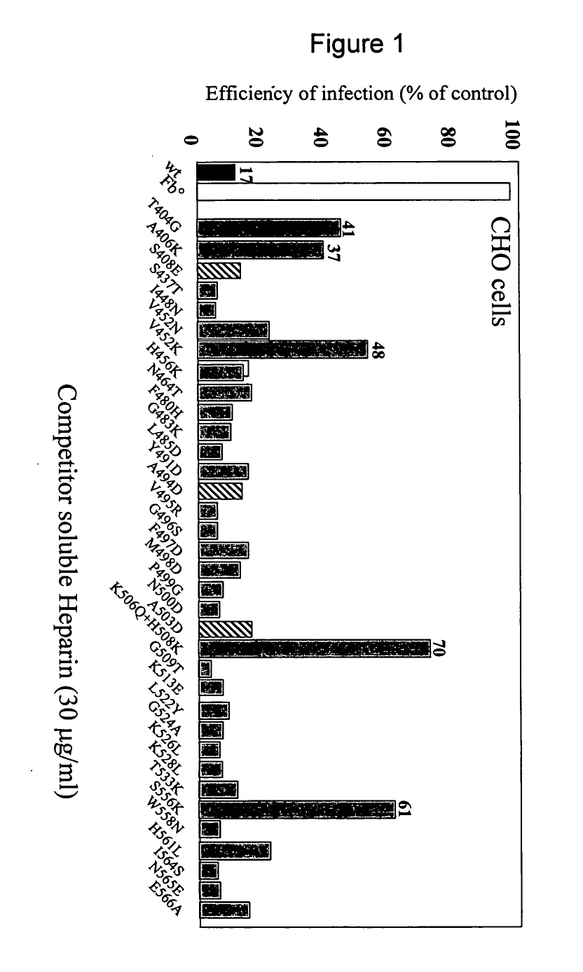

As shown in FIG. 1, infection of CHO cells (CAR−) by the majority of the mutant viruses was greatly reduced in the presence of heparin. Interestingly, five mutant viruses, respectively T404G, A406K, V452K, K506Q/H508K and S555K, were identified which were able to infect CHO cells in the presence of high concentrations of soluble heparin, suggesting that the putative binding of the corresponding viruses to the heparan sulfate was impaired. In marked contrast, for all other mutant adenoviruses including those ablated for CAR binding (e.g. S408E, A494D, A503D, etc.), the infection of the target cells still seemed to be mediated through heparan sulfate suggesting that the corresponding fiber mutations have no significant adverse effects on this pathway. Same results were obtained with increasing concentration of heparin (10, 30, 50 and 100 μg/ml), then suporting specificity of these results. The mutations of the so called Heparan-ablated adenoviruses are located in the AB loop (T404G; A406K), in the CD loop (V452K), in the DG loop (K506Q/H508K), and in the I sheet (S555K).

1.5 Properties of Heparan Mutants Concerning CAR Pathway

The five Heparan-ablated adenoviruses, bearing respectively T404G, A406K, V452K, K506Q/H508K and S555K fiber mutations, were then tested for their ability to infect 293 cells. 5×10⁴ 293 cells were infected with Ad-LacZ, Ad-LacZ/Fb^{o} as controls or with these five fiber-modified mutant viruses at a MOI of 1 IU/cell. At 48 h postinfection, the cells were stained for beta-galactosidase expression.

As a result, the five Heparan-abalted adenovirus still efficiently infects 293 cells through CAR binding, with similar infectivity than that of wild type adenovirus.

Infection was also performed in the presence of recombinant Ad5 knob protein as a competitor of CAR binding, For this purpose, 293 cells were incubated for 30 min at 37° C. with 10 μg/ml of Ad5 knob protein purified from a recombinant E. coli strain. One hundred IU/10⁵ cells of Ad-LacZ (corresponding to, 4×10³ P/10⁵ cells), fiberless Ad-LacZ/Fb^{o} (corresponding to 4×10⁷P/10⁵ cells) or Heparan-ablated mutants expressing LacZ (T404G, A406K V452K, K506Q/H508K and S555K corresponding to 5×10⁴-3×10⁷ P/10⁵ cells), were then added. 24 h post-infection, the cells were stained for β-galactosidase expression. The efficiency of infection was expressed as the percentage of β-galactosidase positive cells in the absence of knob. The number of blue cells counted in the control wells (in the absence of knob) ranges from 100 to 400.

It was shown that the five Heparan-ablated mutant adenovirus are fully competited (between 80 and 90% of inhibition) by preincubating the cells with a saturating concentration of recombinant Ad5 knob, confirming that their corresponding fiber mutations do not impair and interfer with CAR binding.

### Example 2

### Possibility of Retargeting Infection of the Heparan-Ablated Mutant Virus By Addition of a Polylysine Ligand at the C-Terminus of the Fiber

A polysine ligand was inserted at the C-terminus of the modified fiber ablated for HSG binding (T404G, A406K, V452K, K506Q/H508K and S555K mutations respectively) and LacZ-expressing adenoviral particles harboring the 7K retargeted and HSG-ablated fiber were constructed as described in the “Materials and Methods” section. The 7K ligand is composed of seven lysine residues (7K) and is known to confer the ability to efficiently bind heparan sulfate proteoglycans on the surface of target cells. In other terms, addition of the 7K ligand to the HSG ablated fibers will therefore restore HSG binding, and thus, demonstrate the possibility of retargeting adenovirus tropism as desired (by selecting an appropriate ligand).

As a result, 7K-containing mutant viruses have similar properties as their mutant conterparts (devoid of ligand), in terms of growth kinetics, maturation, and yield production. However, their ability to infect cells via heparan sulfate was restored and moreover amplified. These results show that the above HSG-ablated modified fiber can incorporate ligand moieties at the C-terminal extremity of the knob, to target adenovirus infection to desired cell types.

### Example 3

### Constructions of Fiber Mutants Impaired in Both HSG and CAR Pathways of Infection and Properties of These Combinated-Mutant Viruses

Fiber residues involved both in binding to the CAR and HSG receptors were mutated to provide a modified fiber impaired in both pathways. In the context of this invention, any mutation fiber mutations altering CAR binding (e.g. S408E, A503D, A494D) can be combined to any mutation involved in HSG binding (e.g. T404G, A406K, V452K, H506Q, H508K, S555K, H506Q/H508K). The following combinations of mutations altering binding to CAR and HSG receptors and corresponding adenoviral particles were generated as described in the Materials and Methods:

Ser408Glu and Val452Lys (S408E/V452K);

Ser408Glu and Lys506Gln/His508Lys (S408E/K506Q/F508K);

Ser408Glu and Ser555Lys (S408E/S555K);

Ala503Asp and Val452Lys (A503D/V452K);

Ala503Asp Glu and Lys506Gln/His508Lys (A503D/K506Q/H508K); and

Ala503Asp and Ser555Lys (A503D/S555K).

3.1 Incorporation of Modified Fiber in Purified Adenoviral Particles

The effect of the combination of CAR⁻ and HSG⁻ mutations on adenoviral capsid formation was evaluated by Western blotting as described in Example 1 and compared to the incorporation of the wild type Ad5 fiber (Ad-LacZ virus having a wild type fiber as positive control). The various viruses were produced on 293 cells and purified by cesium chloride gradient (density 1.34 g/ml). 2×10¹⁰ purified particles were subjected to a 4-12% Bis-Tris Nupage gel and transferred to nitrocellulose. Filters were hybridized either with sera directed against the Ad5 Knob (provided by Dr. Gerard; Henry et al., 1994, J. Virol. 68, 5239-5246) or with an anti-penton base serum (polyclonal rabbit anti-penton antibody; provided by Pr. Boulanger), and were then treated with a secondary horseradish peroxidase-conjugated donkey anti-rabbit antibody.

A strong positive signal having the expected molecular weight was highlighted for the wt Ad-LacZ virus (positive control) and all the mutated fiber vectors.

These results demonstrate that the combinations of CAR⁻ and HSG⁻ mutations have no deleterious effect on the correct folding of the fiber protein and do not prevent its assembly into the capsid.

3.2 Maturation of Combinated Fiber-Modified Viruses

The protein profile of the CAR⁻ and HSG⁻ mutant adenovirus particles was analyzed and compared to CAR⁻ mutant Ad-LacZ/Fb S408E (which fiber has the mutation Ser408Glu), Ad-LacZ (having the Ad5 wild-type fiber) as positive control, and Ad-LacZ/Fb^{o} (a fiber-deleted Ad5) as negative control. For this purpose, the various viruses were produced on 293 cells and purified on cesium chloride gradient. 2×10¹⁰ purified particles were subjected to a 10% SDS-polyacrylamide gel subsequently revealed by silver staining.

The combined CAR⁻ and HSG⁻ mutant viruses exhibit the same protein profile as the Ad-LacZ control and Ad-LacZ/Fb S408E CAR⁻ mutant. In all cases, the modified fiber proteins are present in the viral particle in stoichiometric amounts as the wild-type adenovirus. On marked contrast, the fiber-deleted Ad-LacZ/Fb^{o} virus still contains precursors of hexon-associated protein (pVI), of minor core protein (pVII) and of pVIII protein, indicative of an incomplete proteolytic processing.

3.3 Growth Characteristics of Fiber-Modified Viruses

Growth properties of the CAR⁻ and HSG⁻ mutant adenoviruses were analysed on 293 cells and compared to the growth of single modified counterparts CAR⁻ mutant Ad-LacZ/Fb S408E, HSG⁻ mutant viruses (Ad-LacZ/Fb V452K, Ad-LacZ/Fb K506Q/H508K, or Ad-LacZ/Fb S555K), and Ad-LacZ positive control. 293 cells were infected at a MOI of 10 particles/cell with the various mutant viruses or controls. Infected cells and supernatant were harvested at 24, 48, 56, 64 and 72 h post-infection and were treated by three freeze-thawing cycles to release virus particles. Titers of released viruses were determined by beta-galactosidase staining.

The propagation of CAR⁻ and HSG⁻ ablated mutant viruses is greatly altered in 293 (CAR+) cells, as evidenced by the poor formation of infectious units even 721 h post-infection. It should be noted that the a poor virus yield is also obtained with the CAR⁻ Ad-LacZ/Fb S408E. In marked contrast, propagation of HSG⁻ mutant viruses Ad-LacZ/Fb V452K, Ad-LacZ/Fb K506Q/H508K, and Ad-LacZ/Fb S555K is efficient and in the same range of that obtained with the wild-type adenovirus Ad-LacZ giving between 8 to 10×10E8 p/ml at 72 h post-infection. These results correlate with the CAR⁻ phenotype of the impaired mutants.

After large-scale production, the titers of infectious mutant virus (IU/ml) was also markedly reduced compared to the titer obtained with adenovirus bearing a wild type fiber. This correlates with the large augmentation of the IU/particle ratio, as illustrated in Table 4.

TABLE 4
Physical characteristics of the fiber mutant viruses

| | Particle/ml | Infectious Unit (IU)/ | |
|---|---|---|---|
| virus | (P) | ml (293 cells) | IU/Particle |
| Ad-LacZ | 2.5 × 10¹² | 1.3 × 10¹¹ | 1/40 |
| Ad-LacZ/Fb-S408E | 8.6 × 10¹² | 8.2 × 10⁸ | 1/10487 |
| Ad-LacZ/Fb- | 1.15 × 10¹² | 1.1 × 10⁸ | 1/10454 |
| S408E/V452K | | | |
| Ad-LacZ/Fb- | 1.88 × 10¹² | 4.7 × 10⁸ | 1/4000 |
| S408E/K506Q/H508K | | | |
| Ad-LacZ/Fb- | 2.1 × 10¹² | 1.34 × 10⁸ | 1/15671 |
| S408E/S555K | | | |

These results are the consequence of the fiber mutations affecting residues involved in CAR and HSG binding, thus hampering the combined CAR⁻ and HSG⁻ mutant adenovirus to entry 293 cells via CAR receptor.

3.4 Properties of Combined Mutant Viruses Concerning CAR Pathway

The infectivity of CAR+293 cells of the combined CAR⁻ and HSG-ablated adenoviruses was evaluated in the presence or in the absence of knob competitor. 5×10⁴ 293 cells were infected with either CAR⁻ and HSG⁻-ablated adenoviruses (Ad-LacZ/Fb-S408E/N452K, Ad-LacZ/Fb-S408E/K506Q/0508K and Ad-LacZ/FbS408E/S555K, respectively) or Ad-LacZ and Ad-LacZ/FbS408E as controls at a MOI of 100 particles/cell. At 24 h post-infection, the cells were stained for beta-galactosidase expression. As mentioned in section 3.3, all CAR⁻ and HSG⁻ mutants do not efficiently infect 293 cells due to thir impairement in CAR binding, similarly to Ad-LacZ/Fb S408E mutant.

Infection was also performed in the presence of recombinant Ad5 knob protein as a competitor of CAR binding. For this purpose, 293 cells were incubated for 30 min at 37° C. with 10 μg/ml of Ad5 knob protein purified from a recombinant E. coli strain before being infected with one hundred particles/cells of Ad-LacZ, CAR-deficient Ad-LacZ/S408E or combined CAR⁻ and HSG⁻ablated mutants expressing LacZ (8408E/V452K; S408E/K506Q/H508K; and S408E/S555K, respectively). 24 h post-infection, the cells were stained for beta-galactosidase expression. The efficiency of infection was expressed as the percentage of beta-galactosidase positive cells in the absence of knob. The number of blue cells counted in the control wells (in the absence of knob) ranges from 100 to 400.

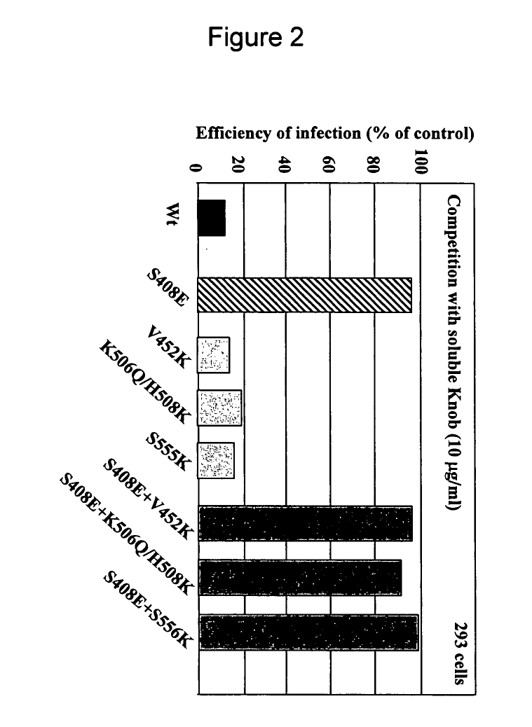

As illustrated in FIG. 2, 293 infection of Ad-LacZ is strongly competited by soluble knob (approximately 90% inhibition) as expected due to the blockage of the CAR pathway by the recombinant knob. As described in Example 1, the HSG− mutants (V452K, K506Q/H508K and S555K) are also strongly competited by preincubating the 293 cells with a saturing concentration of recombinant knob. In marked contrast, the combined CAR⁻ and HSG⁻ ablated mutant adenovirus are not or very poorly competited (between 0 and 10% of inhibition) by recombinant Ad5 knob, as well as the CAR⁻ deficient fiber S408E virus control, confirming their unability to use CAR pathway. These results show that the HSG⁻ mutations (V452K, K506Q/H508K and S555K) do not interfer with the S408E mutation ablating binding to CAR.

3.5 Properties of Combined Mutant Viruses Concerning HSG Pathway

The ability of CAR⁻ and HSG⁻ mutant adenoviruses to bind cellular HSG receptors was evaluated by a competition assay using soluble heparin. CAR deficient CHO cells were infected at a MOI of five hundred particles/cells by either the combined CAR⁻ and HSG⁻ mutant adenovirus or control viruses, the CAR⁻ Ad-LacZ/Fb S408E mutant, the HSG⁻ virus mutants (Ad-LacZ/Fb V452K, Ad-LacZ/Fb K506Q/H508K, and Ad-LacZ/Fb S555K respectively), or the positive control Ad-LacZ (equiped with a wild type fiber). Competition assays were performed by pre-incubating the virus with heparin (30 μg/ml, Sigma) before infecting CHO cells. 48 h post-infection, the cells were stained for beta-galactosidase expression. The efficiency of infection was expresses as the percentage of beta-galactosidase positive cells in the absence of heparin. The number of blue cells counted in the control wells (in the absence of knob) ranges from 100 to 400.

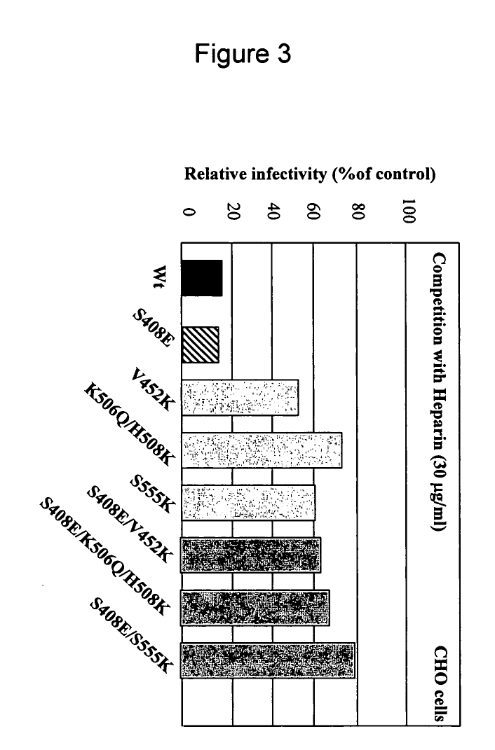

As shown in FIG. 3, infection of CHO cells (CAR−) by Ad-LacZ and Ad/Fb S408E was greatly reduced in the presence of heparin, indicating that the infection of the target cells still seemed to be mediated through heparan. In contrast, the combined mutant viruses, Ad-LacZ (S408E/V452K, S408E/K506Q/H508K and S⁴08E/S555K, respectively) were able to infect CHO cells in the presence of high concentrations of soluble heparin with the same efficiency than in the absence of heparin, suggesting that the putative binding of the corresponding viruses to CHO cells is independent of the heparan sulfate proteoglycans. Same results were obtained with increasing concentration of heparin (10, 30, 50 and 100 μg/ml), then suporting specificity of these results.

These experiments confirm that the combination with CAR⁻ mutation (S408E) do not interfer with the HSG ablating binding V452K, K506Q/H508K and S555K mutations.

The combination of HSG mutation (V452K, K506Q/H508K and S555K) with the CAR⁻ mutation (A503D) can be tested as illustrated in Example 3.

1. A modified adenoviral fiber containing at least one mutation affecting one or more amino acid residue(s) of said adenoviral fiber interacting with at least one glycosaminoglycan and/or sialic acid-containing cellular receptor.

2. The modified adenoviral fiber according to claim 1, wherein said modified adenoviral fiber has an affinity for said glycosaminoglycan or sialic acid-containing cellular receptor of at least about one order of magnitude less than a wild-type adenoviral fiber.

3. The modified adenoviral fiber according to claim 1, wherein said glycosaminoglycan-containing cellular receptor is a heparin- or heparan sulfate-containing cellular receptor.

4. The modified adenoviral fiber according to claim 3, wherein said heparin- or heparan sulfate-containing cellular receptor is a heparan sulfate glycosaminoglycan (HSG) cellular receptor which normally interacts with the wild-type adenoviral fiber to mediate adenovirus attachment to a host cell.

5. The modified adenoviral fiber according to claim 1, wherein said mutation affects one or more amino acid residue(s) within the AB loop, the CD loop, the DG loop and/or the beta sheet I of the knob.

6. The modified adenoviral fiber according to claim 1, wherein said mutation affects one or more amino acid residue(s) selected from the group of residues consisting of the threonine in position 404, the alanine in position 406, the valine in position 452, the lysine in position 506, the histidine in position 508, and the serine in position 555 of the wild type Ad5 fiber protein as shown in SEQ ID NO: 1.

7. The modified adenoviral fiber according to claim 6, wherein said mutation comprises:
The substitution of the threonine in position 404 by glycine,
The substitution of the alanine in position 406 by lysine,
The substitution of the valine in position 452 by lysine,
The substitution of the lysine in position 506 by glutamine,
The substitution of the histidine in position 508 by lysine, or
The substitution of the serine in position 555 by lysine,
Or any combination thereof.

8. The modified adenoviral fiber according to claim 6, wherein said mutation comprises:
the substitution of the lysine in position 506 by glutamine and the substitution of the histidine in position 508 by lysine;
the substitution of the threonine in position 404 by glycine, the substitution of the lysine in position 506 by glutamine and the substitution of the histidine in position 508 by lysine;
the substitution of the alanine in position 406 by lysine, the substitution of the lysine in position 506 by glutamine and the substitution of the histidine in position 508 by lysine;
the substitution of the valine in position 452 by lysine, the substitution of the lysine in position 506 by glutamine and the substitution of the histidine in position 508 by lysine;
the substitution of the lysine in position 506 by glutamine, the substitution of the histidine in position 508 by lysine and the substitution of the serine in position 555 by lysine.

9. The modified adenoviral fiber according to claim 1, wherein said mutation affects one or more amino acid residue(s) selected from the group of residues consisting of the threonine in position 404, the aspartic acid in position 406, the valine in position 452, the lysine in position 506, the glutamine in position 508, and the threonine in position 556 of the wild type Ad2 fiber protein.

10. The modified adenoviral fiber according to claim 1, wherein said modified adenoviral fiber further comprises at least one additional mutation affecting one or more amino acid residue(s) of said adenoviral fiber interacting with the CAR cellular receptor.

11. The modified adenoviral fiber according to claim 10, wherein said modified adenoviral fiber has an affinity for said CAR cellular receptor and said glycosaminoglycan and/or sialic acid-containing cellular receptor of at least about one order of magnitude less than a wild-type adenoviral fiber.

12. The modified adenoviral fiber according to claim 10, wherein said additional mutation affects one or more amino acid residue(s) selected from the group consisting of the serine in position 408, the proline in position 409, the arginine in position 412, the lysine in position 417, the lysine in position 420, the tyrosine in position 477, the arginine in position 481, the leucine in position 485, the tyrosine in position 491, the alanine in position 494, the phenylalanine in position 497, the methionine in position 498, the proline in position 499 and the alanine in position 503 of the wild type Ad5 fiber protein as shown in SEQ ID NO: 1.

13. The modified adenoviral fiber according to claim 12, wherein said additional mutation comprises:
the substitution of the serine in position 408 by glutamic acid (S408E),
the substitution of the proline in position 409 by lysine (P409K),
the substitution of the tyrosine in position 477 by alanine (Y477A),
the substitution of the leucine in position 485 by lysine (L485K),
the substitution of the tyrosine in position 491 by aspartic acid (Y491D),
the substitution of the alanine in position 494 by aspartic acid (A494D),
the substitution of the phenylalanine in position 497 by aspartic acid (F497D),
the substitution of the methionine in position 498 by aspartic acid (M498D),
the substitution of the proline in position 499 by glycine (P499G),
the substitution of the alanine in position 503 by aspartic acid (A503D), or
any combination thereof.

14. The modified adenoviral fiber according to claim 13, wherein said modified adenoviral fiber comprises (i) the substitution of the serine in position 408 by glutamic acid, the substitution of the lysine in position 506 by glutamine and the substitution of the histidine in position 508 by lysine (S408E/K506Q/H508K) (ii) the substitutiton of the alanine in position 503 by aspartic acid, the substitutiton of the lysine in position 506 by glutamine and the substitutiton of the histidine in position 508 by lysine (A503D/K506Q/H508K), (iii) the substitutiton of the serine in position 408 by glutamic acid and the substitutiton of the serine in position 555 by lysine (S408E/S555K), or (iv) the substitutiton of the alanine in position 503 by aspartic acid and the substitutiton of the serine in position 555 by lysine (A503D/S555K).

15. The modified adenoviral fiber according to claim 1, wherein said modified adenoviral fiber trimerizes when produced in a eukaryotic host cell.

16. A trimer comprising the modified adenoviral protein of claim 1.

17. The trimer according to claim 16, having an affinity for a native glycosaminoglycan and/or sialic acid-containing receptor of at least about one order of magnitude less than a wild type adenoviral fiber trimer.

18. The trimer according to claim 16, containing a modified adenoviral fiber, wherein said trimer further has an affinity for a native CAR cellular receptor of at least about one order of magnitude less than a wild type adenoviral fiber trimer.

19. A DNA fragment or expression vector encoding the modified adenoviral fiber of claim 1.

20. An adenoviral particle lacking a wild-type fiber and comprising the trimer of claim 16.

21. The adenoviral particle of claim 20, further comprising one or more penton base having a mutation affecting at least one native RGD sequence.

22. The adenoviral particle of claim 20, further comprising a ligand.

23. The adenoviral particle of claim 22, wherein said ligand binds at least one cell-surface anti-ligand other than a native receptor which normally mediates cell attachment and/or uptake of a wild-type adenovirus.

24. The adenoviral particle of claim 23, wherein said cell surface anti-ligand is selected from the group consisting of cell-specific markers, tissue-specific receptors cellular receptors, antigenic peptides, tumor-associated markers, tumor-specific receptors and disease-specific antigens.

25. The adenoviral particle of claim 22, wherein said ligand is immunologically, chemically or genetically coupled to a viral polypeptide exposed at the surface of said adenoviral particle.

26. The adenoviral particle of claim 25, wherein said viral polypeptide exposed at the surface of said adenoviral particle is selected from the group consisting of penton base, hexon, fiber, protein IX, protein VI and protein IIIa.

27. The adenoviral particle of claim 26, wherein said ligand is genetically inserted in said modified fiber, especially at the C-terminus or within the HI loop.

28. The adenoviral particle of claim 26, wherein said ligand is genetically inserted in the protein pIX, especially at the C-terminus or within the C-terminal portion of said protein pIX.

29. The adenoviral particle of claim 20, which is an empty capsid.

30. The adenoviral particle of claim 20, comprising an adenoviral genome.

31. The adenoviral particle of claim 30, wherein said adenoviral genome is replication-defective.

32. The adenoviral particle of claim 30, wherein said adenoviral genome comprises at least one gene of interest placed under the control of the regulatory elements allowing its expression in a host cell.

33. The adenoviral particle of claim 32, wherein said regulatory elements allowing the expression of said gene of interest are functional within a host cell presenting at its surface an anti-ligand to which said ligand binds.

34. The adenoviral particle of claim 32, wherein said regulatory elements comprise a promoter selected from the group consisting of tissue-specific promoters and tumor-specific promoters.

35. A process for producing the adenoviral particle according to claim 20, comprising the steps of:
Introducing said adenoviral particle or the genome of said adenoviral particle into a suitable cell line,
Culturing said cell line under suitable conditions so as to allow the production of said adenoviral particle, and
Recovering the produced adenoviral particle from the culture of said cell line, and
Optionally purifying said recovered adenoviral particles.

36. The process according to claim 35, wherein said adenoviral particle is replication-defective and said cell line complements at least one defective function of said adenoviral particle.

37. The process of claim 35, wherein said cell line comprises either in a form integrated into the genome or in episome form a DNA fragment or an expression vector encoding the modified adenoviral fiber containing at least one mutation affecting one or more amino acid residue(s) of said adenoviral fiber interacting with at least one glycosaminoglycan and/or sialic acid-containing cellular receptor.

38. The process according to claim 37, wherein said cell line is further capable of complementing one or more adenoviral functions selected from the group consisting of the functions encoded by the E1, E2, E4, L₁, L2, L3, L4, L5 regions or any combination thereof.

39. The process according to claim 37, wherein said cell line is produced from the 293 cell line or from the PER C6 cell line.

40. A composition comprising the adenovirus particle according to claim 20, in combination with a vehicle which is acceptable from a pharmaceutical point of view.

41. The composition of claim 40, wherein said adenovirus particle is conjugated to a lipid or polymer.

42. A method for the preparation of a drug intended for the treatment or the prevention of a disease in a human or animal organism by gene therapy comprising using the adenovirus particle according to claim 20.

43. The method according to claim 42, wherein the disease is a cancer, including glioblastoma, sarcoma, melanomas, mastocytoma, carcinomas as well as breast, prostate, testicular, ovarian, cervix, lung, kidney, bladder, liver, colon, rectum, pancreas, stomac, esophagus, larynx, brain, throat, skin, central nervous system, blood, and bone cancers.
